(19) United States
(12) Patent Application Publication
(10) Pub. No.: US 2011/0027803 A1
(43) Pub. Date: Feb. 3, 2011

(54) Compositions and Methods for Maintenance of Fluid Conducting and Containment Systems

(76) Inventors: Artin Moussavi, London (GB); Catherine Rowley-Williams, Linlithgow (GB); Cameron MacKenzie, Glasgow (GB); Fiona MacKay, Edinburgh (GB); Anne-Marie Fuller, Edinburgh (GB); Vjera Magdalenic, London (GB); Emma Perfect, Edinburgh (GB)

Correspondence Address: OSTRAGER CHONG FLAHERTY & BROITMAN PC, 570 LEXINGTON AVENUE, FLOOR 17, NEW YORK, NY, 10022-6894, US

(21) Appl. No.: 12/808,515

(22) PCT Filed: Dec. 17, 2008
(86) PCT No.: PCT/GB08/04177

§ 371 Date: Aug. 9, 2010

(30) Foreign Application Priority Data
Dec. 17, 2007 (GB) 0724541.8
Oct. 7, 2008 (GB) 0818358.4

(51) Int. Cl. G01N 33/53; G01N 21/00

(52) U.S. Cl. 435/7.9; 436/164

(57) ABSTRACT

Latently detectable small molecules, or ‘labels’, used for monitoring of treatment substances in fluid conducting and containment systems. A composition comprising the treatment substance and the label, a method of manufacturing the composition, a method and kit for use in monitoring the treatment substances in a fluid conducting and containment system, and a method for treating such a system using the composition are also disclosed.

### FIELD OF THE INVENTION

This invention relates to latently detectable small molecules, or ‘labels’, used for monitoring of treatment substances in fluid conducting and containment systems. More specifically, the invention relates to a composition comprising the treatment substance and the label, a method of manufacturing the composition, a method and kit for use in monitoring the treatment substances in a fluid conducting and containment system, and a method for treating such a system using the composition.

### BACKGROUND OF THE INVENTION

Fluid conducting and containment systems are susceptible to inefficiencies and loss of productivity due to damage of component parts. For example, oil and gas operators continue to lose millions of barrels of potential oil production each day due to corrosion, scale and hydrate build up and microbial growth. Systems include, for example, oil and gas reservoirs and their associated infrastructure (wells, pipelines, separation facilities etc), petrochemical processing facilities, refineries, paper manufacture, mining, cooling towers and boilers, water treatment facilities and natural and man-made water systems e.g. lakes, reservoirs, rivers, and geothermal fields.

Keeping equipment and pipes healthy is ultimately the most efficient way to ensure maximum production. The fluid conducting and containment portions of such systems must be continually monitored as many factors can reduce flow efficiency, for example, corrosion of pipes and build up of microbial growth, scale, hydrates, asphaltenes and waxes. Monitoring of natural water systems is also important to provide information on the flow of water, microbial spread, pollution etc. Detectable moeties can be used to monitor the efficiency of flow of fluid and specific components of fluid in systems. Applications include investigation of leaks, speed of flow and how fluid from different systems becomes mixed. The detectable moieties may be associated with treatment substances or microbes as labels so that the distribution of the treatment substances or microbes throughout the system can be monitored. The movement of organisms in systems or in natural environments may be investigated, for example, the movement of algal blooms in the sea that provides information on currents and risk related to pollution.

Treatment substances may be introduced into the fluid in the systems to minimise problems. The term a “treatment substance” is not intended to be limited to the substances to which this patent application refers. The term may include scale inhibitors, both polymeric and phosphonates, corrosion inhibitors, hydrate inhibitors, wax inhibitors, anti-fouling agents, asphaltene inhibitors, hydrogen sulphide scavengers, pH stabilisers, flow additives, anti-foaming agents, detergents and demulsifiers used in oil and gas wells, oil and gas pipelines, petrochemical processing plants, paper manufacture, mining, cooling towers, boilers, water treatment facilities and natural water courses. The concentration of treatment substances must be monitored to ensure that they are maintained at effective concentrations in the fluid conducting and containment systems. The frequency of chemical interventions is a critical cost factor.

The monitoring process can be labour-intensive and expensive, especially in cases requiring monitoring of treatment substances used in off-shore sites such as oil wells (production wells and injection wells). For the latter, samples are often flown onshore for testing, which is especially expensive and time consuming. As fields mature, flights to shore become less frequent, resulting in less comprehensive testing. Risks of well failure are therefore increased and the need for simple offshore testing grows. In general, there is a need for cost-effective, simple, convenient on-site sample testing methods and compositions for use in such methods, in order to monitor the concentration and distribution of treatment substances, microbial growth and flow of fluid in industrial fluid conducting and containment systems as well as natural water systems e.g. rivers. Being able to monitor the distribution of treatment chemicals, microbial growth and fluid flow would achieve a number of objectives. Minimum inhibitory concentrations of treatment substances can be maintained which will reduce the risk of flow assurance problems in pipelines. Efficiency of usage of treatment substances can be improved since they will only be added when required ie when their concentration drops below the minimum inhibitory concentration. Quantitative evidence of treatment substance usage can be provided, with advantages for monitoring of environmental impact of treatment substances. The early detection of flow assurance problems and the implementation of preventative action to minimise the risks of production loss is possible e.g. early planning of squeeze treatments.

As wells age, the composition of produced fluids changes from predominately hydrocarbons to hydrocarbon/acid brine mixtures. These brine mixtures create a more corrosive environment and, with a greater number of older wells in production, corrosion is an increasing problem. Corrosion inhibitors are used to prevent corrosion. Typically oil-field pipeline inhibitors are blends of chemicals which adsorb strongly to the metal surface where they form a permanent barrier layer on the metal surface that prevents attack. Corrosion inhibitors may contain inorganic anions such as phosphates, chlorides, nitrates, and substituted amines, or other organic surfactants. The latter help to generate a protective layer on the pipes.

Labelling and monitoring of corrosion inhibitors would be useful for a number of reasons. First, the corrosion inhibitor market is large and there is a tendency to use generic compounds in corrosion inhibitor formulations. Thus one labeled component could be used in a wide range of products. Secondly, corrosion inhibitor residuals are difficult to detect, with no simple test being available particularly for offshore use. Some progress has been made in determining concentration of components e.g. using ESI-MS. However, detection of corrosion inhibitor residuals remains difficult, particularly offshore. Finally, the impact of better monitoring on regulations would be positive as the current ‘usage equals discharge’ policy is unlikely to hold true due to complex partitioning behaviours of these chemicals.

Some treatment chemicals can be detected directly e.g. with refractive index, fluorescence, UV absorption, ICP-OES, turbidity or with colourimetry following reaction of chemical groups with coloured compounds as is used in the popular hyamine test for scale inhibitors. Problems may arise with such tests from interferences. Interferences may be from the sample, such as the brine, which can interfere with hyamine tests, from background fluorescence, presence of oil and other treatment chemicals. Interferences may also occur when multiple treatment chemicals from different lines become comingled but when analysis requires them to be differentiated. Since they will generate the same signal in fluorometry, colourimetry, ICP-OES etc this is impossible. Labels on the treatment chemicals can be employed to aid this differentiation. In such cases the labels act as a ‘hook’ and can be used, through binding to a biomacromolecule, to ‘fish out’ particular labeled chemicals from the sample and so allow detection of this treatment chemical in the absence of interferences. In this case the labels may themselves be detected, directly or latently, or some signal from the treatment chemical used. For example polymeric scale inhibitors could be tested with traditional methods such as ICP-OES, hyamine test and corrosion inhibitors may be detected with fluorescence, colormetric binding assays. Where the label is biotin then the biomacromolecules captavidin, streptavidin and avidin surfaces may be used to immobilize the biotin labeled treatment chemical prior to subsequent washing and detection.

Treatment substances are effective at minimum inhibitory concentrations (MIC). By monitoring the concentration of the treatment substances, the dosing regiment can be designed to maintain minimum inhibitory concentrations, which will reduce the risk of flow assurance problems in pipelines. The frequency of intervention with a treatment substance is a critical cost factor, and therefore it is of great benefit to the operator if more treatment substances are only added when absolutely required, for example when their concentration is at or has dropped below the (MIC).

For polymeric scale inhibitor materials, determination of concentrations have traditionally been measured by indirect analytical methods i.e. measuring a chemical property (which usually is not unique to the species of interest). These include ICP-OES and turbidimetric (Hyamine 1622). Polymers containing a significant percentage of Phosphorous can be analysed by ICP while those containing little, or no, Phosphorous are usually analysed by the Hyamine 1622 turbidimetric reaction. Both of these methods are routine within the industry and, in clean water systems, are demonstrably accurate and precise down to a few ppm of polymer. However, in samples of produced water from real fields, both methods suffer from a deficiency in that neither method is specific for the polymeric species used in the field.

A problem that is becoming increasingly serious is the lack of adequate methods for the detection of low levels (i.e. minimum inhibitory concentrations MIC) of such treatment substances. This is particularly the case where the fluid from a large number of wells are joined and flow together along a single flow-line thus presenting problems of co-mingled flow interpretation i.e. determining the concentration of specific chemicals from individual wells. This issue is common in the deepwater wells of the Gulf of Mexico and West Africa and it is considered that it will be a growing problem in the future as reductions in steel usage leads to more comingling of lines. For this reason having a suite of labeled treatment substances where each label would relate to a specific chemical coming from a particular well would be extremely beneficial. Especially considering that, because of the difficulty of reaching these wells each treatment can cost many millions of pounds. Labelling treatment substances with different labels can allow more than one well from a multi-well oil or gas producing system to be monitored in parallel.

As mentioned above, a useful method to monitor the flow of fluid and/or the movement of treatment substances and microbes is to use a detectable moiety. These may be added directly to the flow of fluid or associated with a treatment substance or microbe as a label to be monitored. A variety of moieties have been employed for this purpose, for example fluorescent, coloured or radioactive tracers. The concentration of moieties can be detected at a sampling point downstream from the point of addition to the flow of fluid. This provides information on the flow of fluid or the distribution of the treatment substance or microbe and can be used to advise flow management operations. WO 2005/000747, U.S. Pat. No. 6,312,644, U.S. Pat. No. 5,621,995 and U.S. Pat. No. 5,171,450 describe the use of fluorescently detectable labels for scale inhibitors and other water treatment chemicals. However, fluids used in such systems are frequently highly fluorescent e.g. corrosion inhibitors and oil, and therefore the signal-to-background ratio can be poor, necessitating complicated data processing to measure the concentration of the labelled substance or microbe. It would be preferable to have a moiety for use in detection of treatment substances and microbes that addresses the problem of background signal.

U.S. Pat. No. 6,040,406 describes a polymerisable, latently detectable moiety which is converted by a photoactivator into a moiety that absorbs light within a wavelength from 300 to 800 nm. In other words, the method of detection for this moiety is colourimetry, in which a colour change in a sample indicates the presence and concentration of the moiety. Colourimetry is not always appropriate as a method of detection, for example if it is required that a signal from a coloured or opaque sample such as oil or contaminated water be measured.

U.S. Pat. No. 6,218,491 and U.S. Pat. No. 6,251,680 describe water-soluble polymers having amine-thiol terminal moieties incorporated for the attachment of an amine-reactive detectable label. The detectable label is added to a sample taken from a body of fluid in order to analyse the concentration of the water-soluble polymer. The amine thiol terminal moieties suitable are various derivatives of peptides and polypeptides. The problem with the use of such molecules as labels for treatment substances is that under the extreme conditions encountered within oil and water treatment facilities, amino acid polymer-based molecules are unstable. There remains a need for latently detectable labels that are robust to the harsh environment of the kinds of fluid conducting and containment systems discussed herein.

There are three main methods that have been used to attach labels to treatment chemicals for use in the oil, gas and water industries. In one method the chemicals are labeled post polymerisation i.e. to a pre-existing polymer. For example, U.S. Pat. No. 5,128,419 describes how polymers labeled with pendant fluorescent groups are prepared by the (trans)amidation derivatization of pre-existing polymers having carbonyl-type pendant groups. In these cases there is no regional distinction to where the labels are appended; they are conjugated to the polymer in a statistical manner via the very same groups that are responsible for the activity and functionality of the polymer giving a high probability that the performance of the treatment chemical will be affected.

In a second method, the labeled polymer is prepared by co-polymerisation of ethylenically unsaturated “active” monomers with a particular percentage of “non-active” derivatisable monomer such as vinyl benzyl chloride (VBC). The label can be specifically attached to the VBC groups post polymerisation. This is described in International Patent Publication No. WO/2005/001241.

In a third method, labels may be directly incorporated into the treatment chemical polymer's backbone via a co-polymerisation process whereby the labels themselves have pendant vinyl functionalities that allow them to be polymerised in the presence of other “active” monomers. International Patent Nos. WO01/44403, WO01/81654 and WO98/54569 describe the incorporation of fluorescent monomers into treatment polymers.

All of these methods depend on the statistical attachment of the label to the polymer. In other words, a certain amount of detectable label is added to a certain amount of polymer, and it is statistically predicted that each molecule of polymer will carry a certain percentage of label moieties. There are many problems with such predictions. Some polymer moieties may include no label whereas others may include a proportionally high number of labels. There is no specificity to the position of, or number of labels that will be attached to the polymer. As a result, detection of a certain concentration of labels will not necessarily be quantitatively representative of the concentration of molecules of treatment substance. For example, if some molecules have more than one label, and the label is used as an indicator of the presence of treatment chemical, it may appear that there is more treatment chemical than in fact is present. The reverse could occur where some treatment chemical molecules are not labelled with any labels. Furthermore, where the molecular weight of the polymer is lower than 10,000 then the detection of only labeled species will not provide a true representation of the total amount/concentration of labeled and non-labeled chemical.

An example of where this difference in properties would be problematic is when these polymers are applied in an “oilfield squeeze treatment”. During this process the polymers initially adsorb to the formation rock and slowly release from the formation rock over-time. Their return through the pipeline via the produced water is monitored over time to check that the levels of scale inhibitor polymer are at or above the recommended MIC level. If some polymer molecules have attached more labels than others and the measurements obtained using the detection method are proportional to the amount of label present this could lead to inaccuracies in the analysis; particularly if labeled and non-labeled species have slightly different absorptions to formation rock resulting in staggered return of the different polymer species from the oil well. Such analyses are used to inform the design of repeat treatment schedules. As such, where the concentration appears to be lower than in fact it is in reality within the system, the operator will add more treatment chemical and will therefore incur unnecessary costs. Conversely, the treatment chemical concentration may appear higher than it actually is, giving the impression that the well is protected when it is not. This could have very serious flow assurance consequences affecting oil production such as the blocking up of wells or pipes through scale formation.

Additional problems arise due to the functionality of both the polymeric treatment substance and the functionality of the label. When the discussed prior art methods are used, the labels will be incorporated throughout the length of the polymers, since it is not possible to control the location of label incorporation. As a result, the labels may be less detectable or may be less useful for immobilization purposes especially where the label has properties that allow it to be used to extract the entire polymer from a mixture, because polymers can coil, obscuring the label and preventing access to detection molecules or immobilization surfaces. In such a case, even labeled polymer could go undetected, because the detection molecule is obstructed from interacting with the label. This would result in a reading of treatment substance concentration that would be lower than the true concentration.

In addition, the more labels that are present on the polymer, the greater the chance that their presence will affect the properties/function of the polymer. For example, the efficacy of the treatment polymer could be reduced with the result that its minimum inhibitory concentration (MIC) is higher such that a greater amount of treatment substance will be required to provide the same protection to the wells and pipes thus increasing costs. Another cost related issue is that by their vary nature these labels can be relatively expensive compared to the cost of the monomers used to make treatment chemical polymers so it would be more cost effective to have as little label as possible present. In addition, from a regulatory viewpoint, non-labeled polymers that have already been registered would usually require re-registration if these polymers were then labeled and the label content was above a certain threshold, whereas if the label content is below the threshold the polymer would not require a lengthy and costly re-registration process. For these reasons it would be beneficial to have as little label as possible on the polymer.

Unfortunately, it can be difficult to achieve the attachment of a minimal amount of label to a polymer by statistical means. Current methods would simply involve the use of certain percentage of label and a certain percentage of treatment substance that are statistically predicted to lead to a lower number, preferably one, label per polymer molecule. However, attempts to do this can result in a large proportion of non-labeled polymer in a sample. The implications of this are that an assay based on the detection of label moieties could appear to show that levels of polymer in the system are much lower than they in fact are, because the unlabeled polymer will not be detected.

There remains a need for a label that can be used for monitoring of treatment substances or microbes in fluid conducting and containing systems. Preferably, the labels and any method to associate them with particular treatment substances or organisms would have minimal deleterious impact on the activity or movement of treatment substances and on the system being investigated. For oil and gas applications it is desirable that the labels and association methods are stable enough to withstand any the harsh environments such as those of the oil well or gas well, including high temperatures, high pressures, presence of treatment chemicals, oil and high ionic strength solutions. It is preferable that the label is not subject to such problems as poor signal to background ratio.

It is an object of the present invention to provide compositions that seek to address the problems highlighted above.

### DEFINITIONS

A “label” is defined for the purposes of this description as a moiety that interacts specifically with an associated biomacromolecule. The label may be latently detectable, producing a detectable signal on interaction with said associated biomacromolecule.

“Latently detectable” is used within this description to mean that a label is not detectable by a chosen method of detection, until it interacts with the recognition site of a biomacromolecule. The interaction results in a change in the sample, or a change in the biomacromolecule, which can be detected by the chosen method of detection.

A “composition” is defined for the purposes of this description as the detectable treatment substance that results from the association between a treatment substance and label. The association may result in, for example, chemical coupling or other stable association, for example electrostatic attractions.

A “polymer” is defined for the purpose of this description as a macromolecular chain comprising repeating units. These units may be, for example, monomer units of a treatment compound.

A “copolymer” is defined for the purpose of this description as a polymer comprised of repeating units and having at least two different units. These two different units may be, for example, a treatment substance and a label. This can be produced by labelling monomer units of the treatment substance and subsequently polymerising the labelled monomer units. Alternatively, it can be produced by co-polymerising the labels and monomer units together.

A ‘fluid conducting and containment system’ or a ‘system for conduction and containment of fluid’ or ‘fluid system’ refers to any such system that is used in or by industry. This may include natural water systems. The term may also mean those systems used in industries for which efficiency of flow is important in order to achieve high productivity or to maximise effectiveness. The term may also refer to any system that is treated by treatment substances, the treatment substances being used to enhance flow efficiency within the system. Such treatment substances are discussed within this patent specification. Examples of such fluid conducting and containment systems that would benefit from the present invention include oil and gas reservoirs and their associated infrastructure (wells, pipelines, separation facilities etc), petrochemical processing facilities, refineries, paper manufacture, mining, cooling towers and boilers, water treatment facilities and water systems e.g. lakes, reservoirs, rivers, and geothermal fields. As would be understood by the skilled person, such systems tend to be large, but may include small components and in addition, some such systems may be small, such as microfluidic devices.

A “monomer” is defined as a molecule which can undergo polymerization thereby contributing constitutional units to the essential structure of a macromolecule.

An “Active” monomer is defined as monomer whose functional group/s contribute to the functional properties of the polymer. For the purposes of making a polymeric scale inhibitor monomers whose properties aid in preventing scale formation include, but are not limited to, acrylic acid, vinyl sulphonic acid, vinyl sulphonate salts, vinyl phosphonic acid or vinyl phosphonate salts, vinylidine diphosphonic acid or salts thereof, vinyl acetate, methacrylic acid, vinyl alcohol, styrene-p-sulphonic acid and salts there of, acrylamido-2-methylpropanesulphonic acid (AMPS), hydroxylphosphonoacetic acid (HPA), hyphosphorous acids, acrylamides, unsaturated mono or di-carboxylic acids or anhydrides such as maleic anhydride, maleic acid, fumaric acid, itaconic acid, aconitic acid, mesaconic acid, citraconic acid, crotonic acid, isocrotonic acid, angelic acid and tiglic acid.

A “polymer” is defined for the purpose of this description as a macromolecular chain comprising repeating units. These units may be, for example, monomer units of a treatment compound.

A “copolymer” is defined for the purpose of this description as a polymer comprised of repeating units and having at least two different units. These two different units may be, for example, a treatment substance and a label This can be produced by labelling monomer units of the treatment substance and subsequently polymerising the labelled monomer units. Alternatively, it can be produced by co-polymerising the labels and monomer units together.

The “α” end of the polymer is defined as the head end or the end from which the polymer chain grows.

The “ω” end of the polymer is described as the tail end or the end at which the polymer chain is terminated/stops growing at.

“End capping” or “end-capped” a polymer is defined as attaching a functional group or label to the ω or tail end of the polymer.

To those versed in the art it is well known that “controlled/living” polymerizations such as cationic, anionic and ring-opening, nitroxide mediated, ATRP and RAFT are very useful methods for designing polymer structures allowing the preparation of a wide variety of well-defined polymer structures including end-functionalized polymers. There are various methods by a polymer can be terminally labeled.

A “biomacromolecule” is defined for the purposes of this description as a biomacromolecule that includes a site for the specific interaction, binding or displacement of a small molecule, of which a number of non-limiting examples are listed in Table 1. This interaction may be based on conformational or chemical aspects of the label or the associated biomacromolecule. This may also include the binding or interaction of a latently detectable label with a ligand that is already associated with the biomacromolecule, for example displacement of the ligand by the label. The biomacromolecule may be adapted to produce a signal on binding of the tracer, or it may do so due to an innate, pre-existing property of the biomacromolecule. This signal may be chemical, for example production of hydrogen peroxide, or the signal may be light-based. For example a fluorophore could be attached to a biomacromolecule, such as a molecule of streptavidin. Alternatively, the biomacromolecule may produce a signal due to a pre-existing property, for example it may be a photoprotein and emit light, or it may be an enzyme and produce a molecule on interaction with the tracer. Any biomacromolecule known in the art to associate specifically via such a recognition or binding site with a small molecule would fit this definition. The term may include many small molecule-biomacromolecule pairs exist in nature as listed non-exhaustively below:

TABLE 1

| | Biomacromolecule to which the |
|---|---|
| Label | label binds |
| Biotin | Streptavidin or avidin or neutravidin |
| | or captavidin, also mutant variants |
| | and derivatives of these |
| Selenobiotin | Streptavidin or avidin or neutravidin |
| | or captavidin, also mutant variants |
| | and derivatives of these |
| Oxybiotin | Streptavidin or avidin or neutravidin |
| | or captavidin, also mutant variants |
| | and derivatives of these |
| Thiamine | Thiamine binding protein |
| Riboflavin and Riboflavin-5′- | Riboflavin binding protein |
| phosphate | (flavoprotein) |
| Niacin (nicotinic acid) | Nicotinic acid binding protein |
| Pantothenic acid | Pantothenic acid binding protein |
| Citrate | Citrate binding protein |
| Cobalamin | Cobalamin binding protein |
| Folic acid | Folic acid binding protein |
| Ascorbic acid | Ascorbic acid binding protein |
| Retinol | Retinol binding protein |
| Vitamin D, cholecalciferol and | Vitamin D binding protein e.g. |
| calcitriol | group specific protein (Gc), 25- |
| | hydroxylase, vitamin D receptor, |
| | antibodies (such as from DiaSorin) |
| Vitamin E | Vitamin E binding protein |
| Vitamin K | Vitamin K binding protein |
| Glucose and derivatives including 2- | Glucose binding protein including |
| N-acetyl glucosamine, 1-Methyl- | glucose oxidase |
| beta-D-glucopyranoside, 1-Hexyl- | |
| beta-D-glucopyranoside and | |
| derivatives at position 4. | |
| Fructose | Fructose binding protein |
| Maltose | Maltose binding protein |
| Ribose | Ribose binding protein |
| Other sugars, polysaccharides and | Lectins (various) |
| carbohydrates e.g. arabinose, | |
| deoxyribose, lyxose, ribulose, xylose, | |
| xylulose and starch | |
| Chitin | Chitin binding protein |
| D-Luciferin | Luciferase e.g. firefly luciferase, |
| | railroad worm luciferase, click |
| | beetle luciferase |
| Coelenterazine | Coelenterate luciferases e.g. Renilla, |
| | Gaussia and photoproteins e.g. |
| | aequorin and obelin |
| Histidine | Histidine transporter protein |
| Caffeine | Caffeine binding protein |
| Imidazoline | Imidazoline binding protein |
| Steroid hormones e.g. cortisol | Steroid hormone receptors e.g. |
| | cortisol binding protein |
| Chlorpromazine | Chlorpromazine binding protein e.g. |
| | receptors of central nervous system |
| cAMP | cAMP binding protein |
| cortisol | Cortisol binding protein (reference: |
| | Biology of Reproduction, Vol 18, |
| | 834-842) or cortisol antibody as |
| | used conjugated to luciferase marker |
| | (Sensomics) |
| 6-keto-proslabellandins | 6-keto-proslabellandin antibody, |
| | including labelled antibodies such as |
| | aequorin or GFP labelled versions |
| | available from Senseomics |
| Thyroxine | Thyroxine binding proteins |
| | including thyroxine-binding |
| | globulin, transthyretin and albumin |
| Triiodothyronine | Thyroxine binding proteins |
| | including thyroxine-binding |
| | globulin, transthyretin and albumin, |
| | nuclear Triiodothyronine binding |
| | protein (Proc Natl Acad Sci U.S.A. |
| | 1974 October; 71(10): 4042-4046) |
| Anthocyanins | Glutathione S-transferases |
| Cholesterol | Cholesterol binding proteins such as |
| | VIP21/caveolin and cholesterol |
| | oxidase |
| L-gulono-1,4-lactone | L-gulono-1,4-lactone binding |
| | proteins including: Rv1771, |
| | proteins including: Rv1771, |
| | L-gulono-1,4-lactone |
| | dehydrogenase/oxidase |
| Bile acids and salts including cholic | glutathione S-transferases, bile acid |
| acid, chenodeoxycholic acid, | binding proteins such as ileal bile |
| deoxycholic and glycocholate | acid binding proteins, liver fatty |
| | acid-binding proteins |
| eicosanoids (proslabellandins, | Proslabellandin receptors e.g. |
| prostacyclins, the thromboxanes and | PPARg, Prostacyclin receptors e.g. |
| the leukotrienes) | PTGIR; thromboxane receptors e.g. |
| | TXA2 |
| Vitamin C (L-ascorbate) | L-ascorbate binding protein |
| | including L-ascorbate oxidase |
| Galactose and derivatives including | Galactose binding protein including |
| 2-N-acetyl galactose, 1-Methyl-beta- | galactose oxidase |
| D-galactose and 1-octyl-beta-D- | |
| galactose | |
| Xanthine and hypoxanthine | Xanthine oxidase, xanthine |
| | dehydrogenase, |
| | phosphoribosyltransferase, |
| | Xanthine binding RNAs |
| Catecholamines such as epinephrine | catecholamine regulated protein |
| and norepinephrine | (CRP40), catecholamine binding |
| | proteins, adrenergic receptors (alpha |
| | and beta), epinephrine receptor, |
| | norepinephrine receptor |
| Nucleotides (adenine, cytosine, | Nucleotide binding proteins e.g. G |
| guanine, tyrosine, uracil; | proteins, ATP-binding protein |
| monophosphate, diphosphate and | |
| triphosphate forms) | |

### SUMMARY OF THE INVENTION

In a first aspect of the invention, there is provided a composition for treating a system for conduction and containment of fluid, the composition comprising a treatment substance associated with a label, the association between the treatment substance and the label being sufficiently stable that a detectable signal produced due to interaction of the label with a biomacromolecule is representative of the presence of the treatment substance. This composition is ideal for use within industrial and natural systems because it can be easily and conveniently monitored even on-site at off-shore or remote locations by adding a biomacromolecule, and detecting the resulting signal. The user can be sure that any signal that is produced on addition of the biomacromolecule is due to the presence of the composition, firstly because the biomacromolecule has a high specificity for the label and secondly because the biomacromolecule is associated sufficiently with the label. Thus, no signal will be emitted unless the composition is present. A further advantage is that the label is latently detectable. Therefore, no signal will be produced from the sample, even if it contains the composition, until the biomacromolecule is added. In order to detect the signal attributable to the presence of the composition, therefore, a signal measurement can be taken before and after addition of the biomacromolecule, and the former subtracted from the latter. This simple subtraction ensures that any interfering background signal can be easily removed. Sometimes it is necessary to treat the sample to remove background interference such as autofluorescence by addition of chemicals, heat treatment or bleaching. If labels are directly detectable, they may be affected by such treatment and become less detectable, but a latently detectable label on the other hand will advantageously not be affected by such treatment.

Preferably, the label is attached to the treatment substance. This would provide a particularly stable association between the label and the treatment substance so that the detection of the label can be used as a quantitative indicator of the presence of the treatment substance.

Preferably, the label is attached to the terminal end of the treatment substance. A label may be attached to each of both terminal ends of a treatment substance. This is beneficial because a detection system that is based on detection of labels will produce results that accurately reflect the true concentration of treatment substance in the system, because the concentration of the label is the same as the concentration of polymeric treatment substance. A label positioned at the end of the polymer may also be of more use for detection or immobilisation purposes, because it will be less likely to be rendered inaccessible to a detection or immobilisation molecule due to coiling of the polymer. A further advantage of this method is that the terminal attachment of the label may also reduce the impact of said label on the function and activity of the polymeric treatment substance molecule.

Preferably, the label may be conjugated to a polymerisation initiator and the initiator may then act to initiate polymerisation of the polymeric treatment substance. As such, the polymeric treatment chemical is labelled during the synthesis of the polymer from a number of monomer units. The benefit of this process is that the initiator will always be the first unit of the polymer molecule, and therefore the tag will always be at a terminal end of the polymeric treatment substance.

Preferably, the label is conjugated to a transfer agent creating a functional transfer agent and the transfer agent acts to initiate and terminate the polymerisation simultaneously. As such, the polymeric treatment chemical is tagged during synthesis of the polymer at one end by half of the transfer agent and at the other end by the other half of the agent. The benefit of this process is that the transfer agent will always be the first and last unit of the polymer molecule, and therefore the tag will always be located at a terminal end of the polymeric treatment substance.

Preferably, the label is conjugated to an end-capping agent, and the conjugate acts to terminate polymerisation of the polymeric treatment chemical. As such, the polymeric treatment chemical is tagged during synthesis of the polymer from a number of monomer units. The benefit of this process is that the tag will always be the last unit of the polymer molecule, and therefore the tag will always be located at a terminal end of the polymeric treatment substance.

The use of a tag attached to either an initiator or end-capping agent for the conjugation of the tag to the polymeric treatment substance has additional benefits for ease of manufacture, because this method would initiate the polymerisation of the polymeric treatment substance molecule and also tag it in a single step (i.e. tagging during synthesis) rather than two or more steps (i.e. one to synthesise the polymer and the second to tag it). In addition, this method uses fewer different kinds of chemicals, as the tag and initiator or terminating agent are combined in the same molecule. This is beneficial for storage purposes, regulatory or administrative burden for the company and for convenience of the method.

Preferably, the biomacromolecule includes a site for specific interaction with the label. The biomacromolecule and the label may associate as part of molecular signalling complexes in nature. As such, the biomacromolecule is only capable of interacting with the label, so that a signal is only produced if the label, and therefore the composition, is present. This allows for extremely precise detection of the presence of the composition, reducing the likelihood of false positive results. Preferably, the biomacromolecule does not have to be added to the fluid conducting and containment system, so that it is not damaged by the harsh conditions typically present in such systems.

The detectable signal produced due to the interaction between the label and the biomacromolecule may be an optical signal. This may be generated, for example, because the biomacromolecule is conjugated to a fluorophore and the tracer displaces a quencher, so that a fluorescent signal is emitted. Alternatively, the optical signal may be generated directly due to a chemical, conformational or other change in the biomacromolecule, for example if it is a photoprotein that emits light on contact with the label.

The optical signal may be generated on addition of a second molecule to a sample or fluid containing the composition and the biomacromolecule.

Preferably, the treatment substance is capable of maintaining flow efficiency in the fluid conducting and containment system. Many such systems suffer from problems relating to inefficiencies of flow, and consequent loss of productivity. A latently detectable composition directed towards maintaining flow efficiency, which can be easily monitored, would be of great benefit to an operator.

The treatment substance may include scale inhibitors both polymeric and phosphonates, corrosion inhibitors, hydrate inhibitors, wax inhibitors, anti-fouling agents, asphaltene inhibitors, hydrogen sulphide scavengers, pH stabilisers, flow additives, anti-foaming agents, microbes, detergents and demulsifiers. These treatment substances can be used to address the problems that typically affect flow efficiency of systems for fluid conduction and containment. By having a label associated with the treatment substance according to the invention, it is then easy for the operator to detect the composition and check that effective concentrations are being maintained within the large-scale fluid system.

Preferably, the label is a small molecule that is known to interact with a specific biomacromolecule in nature, for example as part of a molecular signalling complex. This may be because the label fits into an ‘interaction’ or ‘active’ site within the biomacromolecule and is capable of creating a temporary or permanent interaction with the site. The interaction may be due to ionic or covalent bonds, electrostatic interactions or any other bonds or forces, but should be sufficiently stable that a there is enough time for the signal produced as a result of the interaction to be detected. As such, the label is only detected on interacting with the biomacromolecule, so that a signal is only produced if the biomacromolecule is present. This allows for extremely precise detection of the presence of the composition, reducing the likelihood of false positive results.

Preferably, the label is selected from the following and derivatives of: vitamins including biotin, selenobiotin or oxybiotin, thiamine, riboflavin, niacin (nicotinic acid), pathothenic acid, citrate, cobalamin, folic acid, ascorbic acid, retinol, vitamins C, D, E or K; luciferin; coelenterazine; chitin; amino acids such as histidine; or monosaccharides, polysaccharides and carbohydrates including arabinose, deoxyribose, lyxose, ribulose, xylose, xylulose, maltose, glucose, fructose, ribose, or trehalose, caffeine, imidazoline, steroid hormones, chlorpromazine and cAMP, cortisol, 6-ketoprostaglandins, thyroxine, triiodothyronine, anthocyanins, cholesterol, L-gulono-1,4-lactone, bile salts including cholic acid, chenodeoxycholic acid, deoxycholic and glycocholate eicosanoids (prostaglandins, prostacyclins, the thromboxanes and the leukotrienes), galactose and derivatives including 2-N-acetyle galactose, 1-methyl-beta-D-galactose, 1-octyl-beta-D-galactose, xanthine and hypoxanthine, catchetolamines such as epinephrine and norepinephrine, nucleotides such as adenine, cytosine, guanine, tyrosine, uracil, monophosphate, in diphosphate and triphosphate forms and preferably the biomacromolecule is selected accordingly to the label used from; avidin and its functional analogues e.g. streptavidin, neutravidin and nitroavidin; thiamine binding-protein; riboflavin binding protein (flavoprotein); nicotinic acid binding protein; pantothenic acid binding protein; citrate binding protein, cobalamin binding protein; folic acid binding protein; ascorbic acid binding protein; retinol binding protein; vitamin D binding protein e.g. group specific protein (Gc); Vitamin E binding protein; Vitamin K binding protein; luciferase; coelenterate luciferase; chitin binding protein; histidine transporter protein; arabinose binding protein; deoxyribose binding protein; lyxose binding protein; ribulose binding protein; xylose binding protein; xylulose binding protein; maltose binding protein; glucose binding protein; fructose binding protein; ribose binding protein; trehalose binding protein or lectin; caffeine binding protein; imidazoline binding protein; steroid hormone receptors; chlorpromazine binding protein; cAMP binding protein; cortisol binding protein; 6-ketoprostaglandin antibody including labelled antibodies such as aqueorin or GFP labelled antibodies; thyroxine binding proteins including thyroxine-binding globulin, transthyretin and albumin; triiodothronine binding protein; glutathione-S-transferases; cholesterol binding proteins such as VIP21/caveolin and cholesterol oxidase; L-gulono-1,4-lactone binding proteins including Rv1771, L-gulono-1,4-lactone dehydrogenase and L-gulono-1,4-lactone oxidase; glutathione Stransferases and bile binding proteins including ileal bile acid binding proteins and liver fatty acid-binding proteins, prostaglandin receptors including PPARg, prostacyclin receptors including PTGIR and thromboxane receptors such as TXA2; L-ascorbate binding protein including L-ascorbate oxidase; galactose binding protein including galactose oxidase, xanthine oxidase, xanthine dehydrogenase, phosphoribosyltransferase, xanthine binding RNAs, catecholamine regulated protein (CRP40), catecholamine binding proteins, adrenergic receptors (alpha and beta), epinephrine receptor, norepinephrine receptor; nucleotide binding proteins such as G proteins and ATP binding proteins respectively. These label biomacromoleculepairs all have the feature that they associate specifically in nature, so that the treatment composition may be detected accurately. By associating these labels with the treatment substance, the biomacromolecule does not have to be added to an industrial fluid conducting and containment system, which is advantageous because it is not exposed to the damaging harsh conditions typically present in such systems. The label, on the other hand, is robust under such conditions. Thus, the detection of the treatment substance can be conducted under conditions that are suitable for correct functioning of the biomacromolecule.

The detectable signal may be detectable, in the presence the biomacromolecule, by a fluorescence detector, luminescence detector, Raman detector, optical microscope, CCD camera, photographic film, fibre-optic device, photometric detector, MEMS device, single photon detector, spectrophotometer, chromatography system or by eye. The person skilled in the art will understand that the method of detection will be selected on the basis of the type of label biomacromoleculepair used for the treatment chemical.

The compositions described hereinabove are of particular use within fluid conducting and containment systems that require high flow efficiency in order to achieve high productivity.

Such systems include, for example, oil and gas reservoirs and their associated infrastructure (wells, pipelines, separation facilities etc), petrochemical processing facilities, refineries, paper manufacture, mining, cooling towers and boilers, water treatment facilities and water systems e.g. lakes, reservoirs, rivers, and geothermal fields. The advantages of this method for these particular systems are numerous. The detectable signal is specifically indicative of the presence of the composition because the signal is only produced if the biomacromolecule has been added and the tracer is present. The reagents are cheap and easy to store on off-shore or remote locations, such as oil fields or drilling rigs. The compositions can be monitored close to the system, preventing time delays in detecting changes in the flow of fluid within the system that might occur if the samples had to be transported before testing. The compositions are especially useful for these because the common problems of signal interference due to contaminants such as treatment chemicals, oil etc are overcome using latently detectable molecules, because a simple background signal subtraction ensures that any signal is attributable to the presence of the composition.

Preferably, the composition will be detectable at a concentration of at least 1 ppb when in the presence of a biomacromolecule. Such a low concentration allows the composition to be detected even at the low levels required to be effective. Therefore, the concentration can be kept as low as is necessary to achieve the treatment effect and less composition will be wasted.

In a second aspect of the invention, a method of manufacturing a composition as hereinabove described is provided, comprising mixing a treatment substance as hereinabove described with a label as hereinabove described to form a reaction mixture and allowing the treatment substance and label to associate, wherein the association that is formed between the treatment substance and the label is sufficiently stable that a detectable signal produced due to interaction of the label with a biomacromolecule is representative of the presence of the treatment substance.

Optionally, the association may be formed by chemically reacting the treatment substance and the label so that they are associated via ionic bonds, covalent bonds, polar interactions, non polar interactions, hydrogen bonding, metallic bonding, π-bonding, aromatic interactions, coordinate bonding or a combination thereof. Optionally, the treatment substance and label may be conjugated together. Optionally, the treatment substance and label may associate via forces such as hydrostatic or electrostatic forces, aromatic interactions, van der waal forces and dipole interactions. Such an association may be particularly strong and as such could be used for example if the composition is to be stored for longer periods of time or may be subjected to unusual conditions that could threaten the stability of the composition. Preferably, any free label will be removed from the reaction mixture after the association has taken place between the label and the treatment substance. This will ensure that any signal that is detected is indeed due to the presence of the composition, and not just to the presence of free label.

Where the treatment substance is polymeric, a monomer unit of the treatment substance can preferably be provided in for forming the association between the label and the treatment substance so that a labelled monomer unit is the product of the association between the label and the treatment substance.

Preferably, the labelled monomer unit is then polymerised to produce a polymeric labelled composition. Using this method, a labelled monomer unit is the product of the reaction step. This feature introduces some flexibility into the method of manufacture. For example, the number of labels to be incorporated into the treatment composition can be controlled, for example to maximise detectability of the treatment composition in the presence of the biomacromolecule where the signal is otherwise weak.

Preferably the labelled monomer units are present in the reaction mixture from 0.01 to 5% molar amount.

Alternatively, where the treatment substance is polymeric, at least one monomer unit of the treatment substance and at least one monomer unit of the label are mixed together so that they are copolymerised to produce a polymeric labelled composition. This copolymerisation approach also allows the molecular weight of the copolymer to be controlled. Thus, scale inhibitor polymers preferably have a weight-average molecular weight of from 500 to 20000 g/mol depending on the polymer units present. This can be determined by those skilled in the art, preferably using size exclusion chromatography/gel permeation chromatography (GPC).

In a third aspect of the invention, a method of monitoring at least one composition as hereinabove described in a fluid conducting and containment system is provided, comprising adding a predetermined amount of the at least one composition to a fluid at a first location in the system, adding a biomacromolecule to a fluid at a second location in the system, said second location being downstream of the first location wherein the predetermined amount of the composition at the first location is sufficient for the concentration of the composition at the second location to be above its detection limit and the concentration of the biomacromolecule is sufficient to produce a detectable change in the fluid due to a specific interaction between the label and the biomacromolecule; measuring the detectable change in the fluid; measuring the detectable change in the fluid; analysing any measured detectable change to determine the concentration of the label at the second location and using the data obtained in order to assess the concentration of the composition at the second location.

This method provides a number of advantages in the detection of treatment substances. In particular, it addresses the problems outlined above that relate to the poor signal to background ratio commonly observed when monitoring treatment substances within fluid conducting and containment systems. The label is latently detectable, and therefore the signal emitted by the fluid could be measured before and after the addition of the biomacromolecule. The signal measured before addition would be subtracted from the signal measured after addition. The difference between the signals would then be attributed to the interaction between the label and the biomacromolecule. Furthermore, the interaction between the biomacromolecule and the label is highly specific and therefore problems with false-positive signals are reduced. This testing method can be performed on site, reducing or replacing the need for expensive transportation of samples, expensive specialist equipment or other complicated and time-consuming practices.

Optionally, a sample may be taken from the second location so that the monitoring is done outside the fluid conducting and containment system. This will be useful, for example, where the biomacromolecule or any other molecules used to generate a signal due to the presence of the composition cannot be added directly to the fluid in the system.

The sample taken may be treated to improve detection of the signal. This may involve concentration of the sample, bleaching to remove background fluorescence, filtration to remove impurities or immobilisation or extraction. This may improve the detectability of the signal resulting from the interaction between the label and the associated biomacromolecule. This may be especially useful where there is high background fluorescence, other interfering chemicals, or where the signal from the label itself is known to be difficult to detect.

The detectable change may be an optical signal. The signal may be fluorescent, luminescent signal or a colour change, or may be a spectroscopic change such as an altered raman signature. Where the signal is luminescent, spectroscopic or a colour change, autofluorescence from the sample (for example from oil or other contaminants), would not create background noise during measurement of the signal due to the composition in the sample.

The method may further include the step of adding a second molecule to the sample after or simultaneously with the addition of the biomacromolecule to the sample. This will be useful where the change induced in the sample as a result of the interaction between the label and the biomacromolecule is a chemical change. The second molecule could interact with the chemical product and produce a signal. Detection of a particular chemical moiety in a sample in this way is a very simple and convenient method for assessing whether the interaction has taken place. As the interaction can only take place when both the biomacromolecule and the label is present, the presence and/or concentration of the composition will be easy to determine.

The chemical may be hydrogen peroxide. The second molecule may be 10-acetyl-3,7-dihydroxyphenoxazine (ADHP, Amplex® Red) which, in the presence of peroxidase, generates the highly fluorescent product resorufin. The fluorescence emitted from the sample due to the presence of this highly fluorescent product may then be detected and attributed to the presence of the composition. Any background fluorescence may be measured before addition of the second molecule and enzymes, and this measurement subtracted from the measurement of fluorescence after addition of the second molecule and enzyme.

The second molecule may alternatively be Phenol Red which would be added with peroxidase. The Phenol Red would undergo a change in absorbance at 610 nm in the presence of the hydrogen peroxide and peroxidase. A colorimetric assay such as this is particularly useful where the sample fluid is colourless, or where the colour produced during the assay is different to that of the sample fluid. The colour signal is indicative of the presence of the treatment composition in the sample.

The second molecule may alternatively be ferrous ions which are oxidised to ferric ions in the presence of hydrogen peroxide and which interact with the indicator dye xylenol orange to produce a purple coloured complex measureable at 560-590 nm. Optionally, sorbitol may be included in the reaction mixture to amplify the color intensity.

The second molecule may be a cyclic diacyl hydrazides such as luminol. Such molecules are converted to an excited intermediate dianon in the presence of hydrogen peroxide and horseradish peroxidase. This dianion emits light on return to its ground state. Phenols can be used to enhance the reaction up to 1000-fold.

Multiple compositions may be monitored, each composition containing a different treatment substance, each treatment substance having a different label so that each different composition can be differentiated according to [[a]] different signals. This allows the user to detect different types of treatment substances using the different signals, conveniently and in one assay. This is a simple and efficient method of assessing the concentration of many treatment substances within a fluid system, and may be especially useful where the relative proportions of treatment substances at a given time is important for efficacy. If these different substances are assessed at different times, using different experiments, inaccuracies and time delays may occur in this assessment so that the relative proportions cannot be calculated.

The optical signal is preferably detectable by a fluorescence detector, luminescence detector, Raman detector, optical microscope, CCD camera, photographic film, fibre-optic device, photometric detector, MEMS device, single photon detector, spectrophotometer, chromatography system or by eye.

Optionally, the monitoring method can be performed off line. An off-line method allows the user to take a sample from a fluid conducting and containment system, and analyse it at a later stage. Such a system is useful where a sample has been taken from an off-shore oil rig, and the oil rig has become too hazardous for carrying out assessment of the sample. In such cases, the equipment and personnel for analysis of the sample may be located far from the location at which the sample is taken.

Optionally, the monitoring method can be performed inline. An in-line method could involve the use of a loop diverting a small but representative sample volume of fluid from the main flow. The biomacromolecule could be injected into the loop, the sample could then feed into a flow cell and the signal detected by, for example, a snapshot imager or by fluorescence reading. An in-line method would advantageously provide the user with real-time data reflecting the composition of the multiphase sample. In line methods of analysis are preferable to other methods because they provide the means for real-time monitoring of samples that are as representative as possible of the situation in the fluid system. An in line method allows frequent, real-time monitoring as samples do not have to be collected from the bulk flow of the fluid system. In addition, the fluid system does not need to be shut down in order to conduct the monitoring tests.

Optionally, the monitoring method may be performed at-line. An at-line method allows the user to remove a sample from the main flow of the system and analyse it on site, close to the fluid conducting and containment system. This monitoring method is not real time but is rapid, and all of the equipment is portable and may be automated, making this method of testing suitable for offshore use. It may be useful to employ such a method when a biomacromolecule cannot be added to an inline loop in the case that conditions are detrimental to the functionality of the biomacromolecule. In addition, the fluid system does not need to be shut down in order to conduct the monitoring tests.

Optionally, the monitoring method may be performed online. An online method may be an automated monitoring method, which feeds directly into a computerised system for monitoring offsite. For example, an online system may incorporate an automated in-line loop, information from the in-line loop being recorded directly to the operator's computer system so that technicians at a different location may review it. This method advantageously allows data to be recorded in real time, but the personnel required to analyse the data would not need to be on-site. Online monitoring has a number of advantages; no manual handling of the sample is required, there is an immediate response (<1 second) and the result can be correlated to a recognised standard reference method. This monitoring method could be used to provide information where the biomacromolecule is added directly to the flow of fluid, and the signal resulting from an interaction with the label is recorded by an online detector. In addition, the fluid system does not need to be shut down in order to conduct the monitoring tests.

The method of monitoring described hereinabove is of particular use within fluid conducting and containment systems that require high flow efficiency in order to achieve high productivity.

Such systems include, for example, oil and gas reservoirs and their associated infrastructure (wells, pipelines, separation facilities etc), petrochemical processing facilities, refineries, paper manufacture, mining, cooling towers and boilers, water treatment facilities and water systems e.g. lakes, reservoirs, rivers, and geothermal fields.

In a fourth aspect of the invention, there is provided a method of treating a fluid conducting and containment system comprising the steps of determining the concentration of a composition as hereinabove described using the method of monitoring as hereinabove described and administering the at least one composition in order to maintain effective concentrations of said composition for treatment of the system.

This method provides a convenient, simple and quick treatment of a fluid conducting or containment system. The composition, containing the treatment substance and label, is so easily detectable that the process of monitoring and maintaining effective concentrations of the treatment substance in order to treat the system is simplified. No expensive, complicated or sensitive equipment is required. In addition, because the method of monitoring and of treatment can be carried out at the site of the system, there is no time delay in administering more treatment substance if necessary. Therefore, problems such as a build up of scale or corrosion while the treatment substances are at less than effective concentrations will not be exacerbated due to a time delay in processing samples. This treatment method is also particularly useful because waste of treatment substances is reduced (because the user will only add more treatment substance when necessary), and effective concentrations of treatment compounds can therefore be maintained in a more cost-effective manner than would be achieved by arbitrarily or regularly adding more treatment substance. The method of treatment allows early detection of usage of treatment substances and administration of more treatment substance to minimise risks of production losses. The method can also be advantageously used to provide quantitative evidence of treatment substance usage, with advantages for monitoring of environmental impact of treatment substances.

The treatment substances to be monitored and/or administered may be effective in maintaining efficient flow within a fluid conducting and containment system. These treatment substances may be, for example, polymeric scale inhibitors, phosphonate scale inhibitors, corrosion inhibitors, hydrate inhibitors, wax inhibitors, anti-fouling agents, asphaltene inhibitors, hydrogen sulphide scavengers, pH stabilisers, flow additives, anti-foaming agents, detergents and demulsifiers, or a combination thereof.

The method of treatment described hereinabove is of particular use within fluid conducting and containment systems that require high flow efficiency in order to achieve high productivity.

Such systems include, for example, oil and gas reservoirs and their associated infrastructure (wells, pipelines, separation facilities etc), petrochemical processing facilities, refineries, paper manufacture, mining, cooling towers and boilers, water treatment facilities and water systems e.g. lakes, reservoirs, rivers, and geothermal fields. The method is especially useful within such systems for a number of reasons relating to problems with interference due to contaminants such as treatment chemicals, oil etc. The method ensures that a detectable change only occurs in the sample subsequent to addition of the biomacromolecule. Therefore, a simple background signal subtraction will allow detection of the treatment chemical in question.

In a fifth embodiment of the invention, there is provided a kit for use in monitoring at least one composition as hereinabove described in a system for conduction and containment of fluid, comprising a composition as hereinabove described and a biomacromolecule selected accordingly to the label included in the composition. The kit may further including means for taking a sample from said system.

The kit may further including a second detection molecule. This would be convenient if the interaction between the tracer and the biomacromolecule leads to a chemical change in the sample. The second detection molecule could then interact with the chemical product and produce a detectable signal.

The kit may also include an optical detector selected from a fluorescence detector, luminescence detector, Raman detector, optical microscope, CCD camera, photographic film, fibre-optic device, photometric detector, MEMS device, single photon detector, spectrophotometer or chromatography system.

### BRIEF DESCRIPTION OF THE DRAWINGS

A number of embodiments of the invention will now be described, reference being made to examples, experimental data and accompanying figures in which:—

FIG. 1 is a graph showing the conductivity of the permeate following a chemical reaction between biotin ethylenediamine and a carboxylic acid-containing polymeric scale inhibitor, using EDC chemistries;

FIG. 2A is a graph showing the concentration of biotin in permeates following a chemical reaction between biotin ethylenediamine and a carboxylic acid-containing polymeric scale inhibitor, using EDC chemistries;

FIG. 2B is a graph showing a 1:1 ratio of PAA to biotin, as determined using ¹H-NMR (300 MHz) of biotin-labeled PAA in DMSO-d₆

FIG. 2C is a graph showing that the Biotective assay (Fluoreporter assay, Invitrogen) can be used to detect biotin-labeled PAA.

FIG. 3 is a graph showing the activity of unlabeled and biotin labeled scale inhibitor chemicals in a static bottle test, showing results for a buffered control, unlabeled inhibitor in buffer Aqueous control, unlabeled inhibitor in water LUX10/4-1; 1.01:1, labeled inhibitor in buffer 1:1 incorporation biotin:inhibitor LUX10/4-2; 1.76:1, labeled inhibitor in buffer 1.76:1 incorporation biotin:inhibitor LUX10/4-3; 2.07:1 and labeled inhibitor in buffer 2.07:1 incorporation biotin:inhibitor;

FIG. 4 is a graph showing the limit of detection (LOD) of biotin-labeled scale inhibitor;

FIG. 5 shows proton NMR spectra (400 MHz) indicating peaks detected due to the presence of biotin D₂O;

FIG. 6 shows proton NMR spectra (400 MHz) indicating peaks due to the presence of biotin-labeled chemicals prepared according to the invention;

FIG. 7 is a size exclusion chromatogram showing data for biotin in water, biotin NH₂ (biotin ethylenediamine) in water, scale inhibitor polymer in water, labeled scale inhibitor polymer (60 days old in 10 nM MES buffer, pH6) and labeled scale inhibitor polymer (7 days old in water);

FIG. 8 is a graph showing the effect of biotin labelling on the partitioning behaviour of scale inhibitor;

FIG. 9 is a graph showing the robustness of biotin to increasing temperatures at various concentrations;

FIG. 10 is a graph showing excitation and emission spectra of 0.1 mg/cm3 fluorescein and the oil fraction from Miller field produced fluids, diluted to 0.1% in petroleum ether (non-fluorescent);

FIG. 11a is a graph showing the fluorescence detected from various concentrations of biotin in deionised water or 0.1% oil;

FIG. 11b is a graph showing the fluorescence of various concentrations of fluorescein in deionised water or 0.1% oil;

FIG. 12 is a graph showing the fluorescence of label (either 0.8 μM biotin or 0.1 mg/cm3 fluorescein) when mixed with 1%, 0.1%, 0.01% of oil;

FIG. 13 is a graph showing the fluorescence of a solution of GFP (0.1 mg/ml Renilla reniformis protein, 80%, in water) with added biotin, (a) no treatment (b) heat treated (samples were heated to 100° C. for 1 hour in an oven);

FIG. 14 is a graph showing a calibration curve for a range of glucose concentrations. The inset shows a linear fit (R²=0.9979) of the data points for concentrations 0-4.5 ppm;

FIG. 15 is a graph showing a comparison between glucose samples prepared in synthetic formation water and the calibration curve, which was generated using aqueous glucose samples;

FIG. 16 is a graph showing the effects of scale inhibitor 8017C and corrosion inhibitor EC1440A on the concentration of glucose detected. The graph shows the average of duplicate samples;

FIG. 17 is a graph showing results from the glucose assay when carried out in the presence of various concentrations of methanol, IPA and MEG. An aqueous glucose control sample with no added solvent gave a fluorescence reading of 80,227;

FIG. 18 is a graph showing the detectability of glucose in the presence of biotin;

FIG. 19 is a set of graphs showing the stability of glucose at 100, 120 and 150° C. in water and formation water at neutral and low pH;

FIG. 20 is a graph showing the effect of crude oil on the glucose assay. Control (water plus glucose) fluorescence value 78,492;

FIG. 21A is a set of two graphs showing a calibration curve for galactose concentrations of 50, 40, 30, 20, 10, 5, 2.5, 1.25, 0.625, 0.3125 and 0 ppm, and also a linear fit (R²=0.998) of the data points for concentrations 0-10 ppm is shown;

FIG. 21B is a graph showing the results of analysis of the calibration curve samples (0-50 ppm) on three different days with fresh assay reagents prepared each day. The error bars represent 95% confidence intervals;

FIG. 22 is a graph showing a range of concentrations of galactose derivatives were analysed and the fluorescence values compared to those for galactose;

FIG. 23 is a set of graphs showing the effect of various interferences on the galactose assay;

FIG. 24 is a graph showing the results of an assay on various concentrations of fructose, mannose and glucose to determine whether other monosaccharides could be oxidised by galactose oxidase;

FIG. 25 is a graph showing the stability of galactose and octyl-β-galactose at 25, 100 and 120° C. in water and formation water at pH 6-7 and pH 2. The error bars represent 95% confidence intervals from triplicate samples;

FIG. 26 is a graph showing a calibration curve for xanthine concentrations of 50, 40, 20, 10, 5, 2.5, 1.25, 0.625, 0.3125, 0.15625 and 0 ppm. The inset has zoomed in on the lower concentration region;

FIG. 27 is a graph showing a calibration curve for hypoxanthine concentrations of 75, 50, 25, 12.5, 6.25, 3.125, 1.5625, 0.78125, 0.3906, 0.1953, 0.0977, 0.0488, 0.0244, 0.0122 and 0 ppm. The inset has zoomed in on the lower concentration region;

FIG. 28 is a set of graphs showing the effect of various interferences on the xanthine and hypoxanthine assay;

FIG. 29 is a graph showing the stability of xanthine and hypoxanthine at 25 and 120° C. at pH 6-7 and pH 2. The error bars represent 95% confidence intervals from triplicate samples;

FIG. 30A shows schematic diagrams of the structure of labeled and unlabeled corrosion inhibitors;

FIG. 30B is a graph showing results of mass spectrometry analysis of labeled inhibitor showing expected increase in size on labelling.

### DESCRIPTION OF PREFERRED EMBODIMENTS

### Example 1

### Coupling of Biotin to a Polymeric Scale Inhibitor

In order to produce a treatment composition comprising a label and treatment substance according to the invention, the coupling of biotin to a polymeric scale inhibitor was investigated. In one example, an amide bond is formed between biotin ethylenediamine and carboxylic acid-containing polymeric scale inhibitor, using EDC chemistries. This reaction may be performed by those skilled in the art. 1-Ethyl-3-[3-dimethylaminopropyl]carbodiimide hydrochloride (EDC or EDC) is the main water-soluble carbodiimide available and is used to couple carboxyl groups to primary amines. EDC reacts with a carboxyl to form an amine-reactive O-acylisourea intermediate. In the presence of biotin ethylenediamine an amide bond is formed between the carboxylic acid-containing treatment chemical and biotin label. NHS (defined below) is added to stabilize the intermediate increasing the efficiency of the coupling. The small molecule marker used was biotin ethylene diamine The treatment substance was polymeric scale inhibitor, a copolymer of polyvinyl/polysuphonic/poly carboxylic acid, part sodium salt, (pH 5.5). The activity of the treatment substance is 30% and its molecular weight distribution is 1500-2000. The bond formed was amide.

The biotin-labelled polymeric scale inhibitor is purified using size exclusion of ultrafiltration methods, as known to those skilled in the art. Modifications to the protocol, as known to those skilled in the art, can be made to provide labelled chemical in a particular buffer, to provide large quantities, to concentrate using ultrafiltration or to provide a particular ratio of label to treatment chemical.

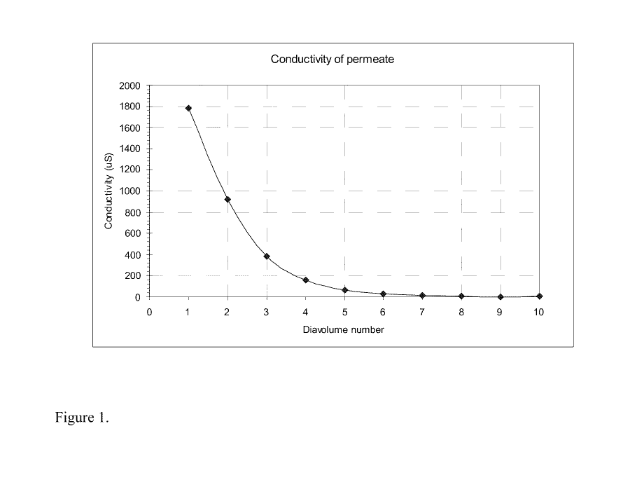

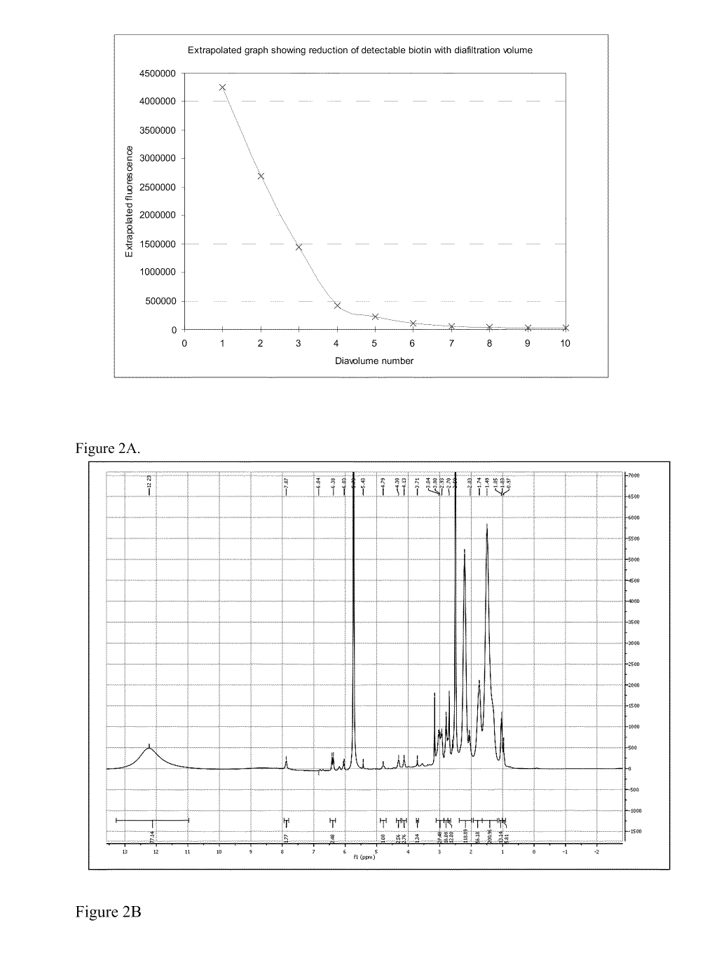

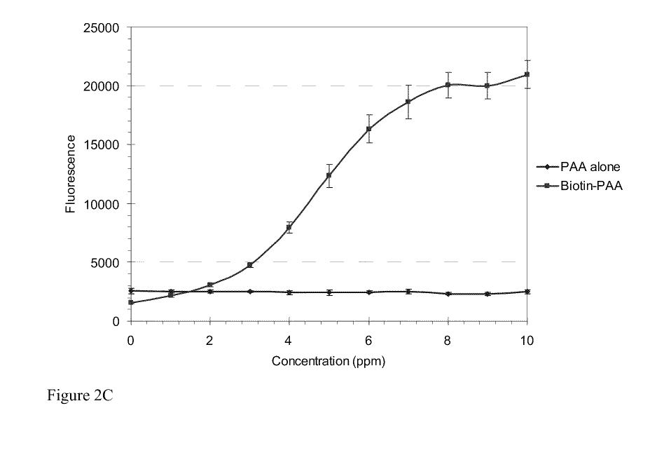

The biotin-labelled treatment substance can be detected following addition of a second reagent. Preferably, the Fluoreporter assay (Invitrogen) was used to detect the concentration of biotin in samples. It was used according to manufacturers instructions. A standard curve was first generated to enable quantification of the amount of biotin in each sample. The conductivity (FIG. 1) and concentration (FIG. 2) of biotin in permeate was determined and results suggest that unreacted biotin is successfully removed. Additionally, the concentration of biotin in the sample of labeled scale inhibitor was determined Theoretical calculations of the expected amount of biotin present were compared with that detected and biotin was detected as expected suggesting no loss of detectability following labelling.

The method of manufacturing the treatment composition comprises providing a latently-detectable label and performing a reaction between the label and the treatment substances to produce a treatment substance-label conjugate. The label may be chemically coupled to a treatment substance or microbe. Where the label is coupled to a treatment substance, the treatment substance may be “finished” or the label may be incorporated during the synthesis of said treatment substance. For the purposes of this description, a “finished” treatment substance is one for which the synthesis reaction has been completed and for which formulation may still be required.

In a first instance, the label may be attached to a “finished” treatment substance. For the purposes of this description, a “finished” treatment substance is one for which the synthesis reaction has been completed and for which formulation may still be required. The label may be attached via a bond or interaction of suitable strength preferably a covalent bond, dative, hydrogen or hydrophobic force. Where the bond formed is a covalent bond this may include, but is not limited to the following bond types, ester, amide, ether, amine, triazole, alkene, alkyne, alkyl, ketone.

In the second instance the label may be incorporated during the synthesis of said treatment substance. This may be achieved by copolymerisation of both monomer units of labels and treatment chemicals approach or by polymerising labelled monomer units of treatment chemical. The label may be attached via a bond or interaction of suitable strength preferably a covalent bond, dative, hydrogen or hydrophobic force. Where the bond formed is a covalent bond this may include, but is not limited to the following bond types, ester, amide, ether, amine, triazole, alkene, alkyne, alkyl, ketone. One advantage of a copolymerisation approach is that active groups of the treatment substance need not be used for coupling, which should ensure the highest possible activity of the treatment substance. It may also allow chemically stronger bonds to be formed e.g. carbon-carbon bonds which may prove more resilient e.g. to high temperatures and pressures. Further, not all treatment substances will be amenable to direct coupling to the label, for example due to the positioning of functional groups.

For treatment substances containing carboxylic acid groups, such as scale inhibitors, a number of chemical reactions can be performed to covalently attach the label. Labels may be conjugated through carboxylic acid groups of the treatment substance and amine groups on the marker. In aqueous systems 1-Ethyl-3-[3-dimethylaminopropyl]carbodiimide hydrochloride (EDC)/N-hydroxysuccinimide (NHS) chemistry may be used. 1-Ethyl-3-[3-dimethylaminopropyl]carbodiimide hydrochloride (EDC/EDAC/EDCI) may be used to couple carboxyl groups to primary amines EDC reacts with a carboxyl group on the scale inhibitor, forming an amine-reactive O-acylisourea intermediate. This intermediate may react with an amine on the marker, yielding a conjugate of the two molecules joined by a stable amide bond. Sulfo-NHS is added to stabilize the intermediate increasing the efficiency of the coupling.

Coupling agents other than EDC may be used and include DEPBT (3-(Diethoxy-phosphoryloxy)-3Hbenzo[d][1,2,3]triazin-4-one), HATU (2-(1H-7-Azabenzotriazol-1-yl)-1,1,3,3-tetramethyl uronium hexafluorophosphate Methanaminium), HBTU (O-Benzotriazole-N,N,N′,N′-tetramethyl-uronium-hexafluoro-phosphate), DCC (Dicyclohexylcarbodiimide), BOP(N-nitrosobis(2-oxopropyl)-amine), DEPC (Diethyl pyrocarbonate), DPPA (bis(diphenylphosphino)amine). In organic solvents 1-hydroxybenzotriazole (HOBt) active esters may be formed. Any number of labels may be conjugated to the treatment substance, in other words multiple markers may be added to one polymer. However, as these carboxylic acid groups may be responsible in part for the activity of the scale inhibitor it may be preferable to couple one marker to each polymer molecule. One way of manipulating the actual number is to adjust the label to treatment substance ratio. Proteins and peptides usually have amines available for this reaction. Alternatively, markers may be functionalised to provide suitable amine groups e.g. biotin ethylenediamine, biotin cadaverine. Biotin ethylene diamine and biotin cadaverine are commercially available products. An alternative strategy for chemically reacting the finished treatment substance to the label is to alter the carboxylic acid prior to coupling so that the variety of useful reactivities can be increased. This can include oxidation, reduction, halogenation, thiolation or any functional group interconversion.

For treatment chemical containing amine groups, such as many corrosion inhibitors, amine groups may be chemically reacted with free carboxylic acid groups on the label. This uses the same chemical techniques as reacting labels to the scale inhibitor chemicals as described previously, such as amide-bond formation chemistry. Corrosion inhibitors may also be chemically reacted with the small molecule marker across the double bond present in the alkyl chain of many aliphatic corrision inhibitors.

Other methods of attaching labels to treatment substances or microbes include, but are not limited to creating the following bond types ester, amide, ether, amine, triazole, alkene, alkyne, alkyl, ketone and carbon-carbon bonds. These bonds may be formed, but are not limited to the following reaction with an alcohol, tert-butyl ether or diazo moiety on a marker to form an ester bond, ester formation by alkylation of the corresponding carboxylic acid salt, amide bond formation via an activated ester with subsequent derivatisation by an amine, addition across an available alkene to form a carboxylic ester, reactions with alkynes to form enol esters or acylals, reaction with another carboxylic acid to form an anhydride, and carbon-carbon bonds can be used to covalently link the two molecules by using a range of available homoreactions or cross coupling reactions Amine groups on corrosion inhibitors may be chemically reacted with free carboxylic acid groups on the label. This uses the same chemical techniques as coupling labels to the scale inhibitor chemicals; i.e. aqueous amide-bond formation chemistry. Many amine-reactive labelling substances are available, as this technology is used to label marker molecules to proteins, which have free primary amine sites available. Corrosion inhibitors may also be chemically reacted with the small molecule marker across the double bond present in the alkyl chain of many aliphatic corrision inhibitors.

An alternative strategy for chemically reacting the finished treatment substance to the label is to alter the carboxylic acid prior to coupling so that the variety of useful reactivities can be increased. This can include oxidation, reduction, halogenation, thiolation or any functional group interconversion. A particularly powerful route to further bond formation is via the ever-expanding range of homoreactions or cross-coupling reactions that can be used to covalently link two molecules.

Where treatment substances contain free sulphonic acid/sulphonate groups, such as scale inhibitors, a label can be covalently linked through the formation of sulphonic esters, sulphonamides or by functional group interchanges. Where treatment substances contain phosphates or phosphoric acids these can be esterified or otherwise coupled and can be made selectively reactive via formation of organophosphate intermediates in order to couple the label to the treatment substance. Where treatment substances contain amines, such as some corrosion and low dose hydrate inhibitors, amide bond formation is possible via an activated ester with subsequent derivatisation by an amine. In this case the label may contain carboxylic acid groups. Many corrosion inhibitors are synthesised from other chemicals e.g. amines are used as building blocks of quaternary amines and imidazoles. Labels functionalised with amine groups may be used during the synthesis of the finished products and so enable incorporation of the label to the treatment substance.

As mentioned above, the label may alternatively be incorporated during the synthesis of a treatment substance. For example, for corrosion inhibitor manufacture during the reaction between fatty acid and amine-containing chemical such as an alkyl amine. Alternatively during the reaction to synthesise phosphonates. Also, the label may be incorporated during polymerisation of a polymeric treatment substance, such as scale or low dose hydrate inhibitors. Many scale inhibitors are polymers, for example but not limited to, phosphino poly carboxylic acid and copolymers of poly vinyl/poly sulphonic/poly carboxylic acid. Low dose hydrate inhibitors are frequently medium to high molecular weight polymers with small repeating units. Various monomer types are in use in these chemicals, including vinyl pyrrolidone, vinyl caprolactam and alkylacrylamide. The label could be attached to a monomer unit of a polymer treatment substance to produce a polymerisable monomer-label conjugate which is then copolymerised to produce a labelled treatment polymer. Alternatively, the label could be reacted during the synthesis of a monomer to produce a labelled-monomer unit which could be copolymerised with the monomer units of the unlabelled treatment substance to produce a labelled treatment polymer. The ratio of labelled and unlabelled monomer used in the copolymerisation can be altered depending on the monomers used and detection sensitivity required.

The advantage of this copolymerisation approach is that active groups of the treatment substance need not be used for coupling, which should ensure the highest possible activity of the treatment substance. It may also allow chemically stronger bonds to be formed e.g. carbon-carbon bonds which may prove more resilient e.g. to high temperatures and pressures. Further, not all treatment substances will be amenable to direct coupling to the label, for example due to the positioning of functional groups.

For incorporation during synthesis of the treatment substance, the label would be appropriately derivatised with one or more chemical functionalities that can either undergo step-growth polymerization or chain-growth polymerisation such as, but not limited to, alkene, alkenyl chloride, alkyne, thiophene, amine, carboxylic acid, alcohol, isocyanate and nitrile. The step-growth polymerization reactions are commonly condensation reactions with bi-functional monomers, for example the synthesis of nylon by reaction of a diamine with a dicarboxylic acid. Chain-growth polymerizations occur via various mechanisms such as radical, anionic and cationic polymerisations usually requiring an initiator and coordination involving a transition metal catalyst. This type of polymerization usually results in carbon-carbon bond formation. The most frequently used monomers in chain-growth polymerizations contain vinyl groups.

Further examples of methods for coupling the labels to the treatment substances are detailed as follows. Riboflavin can be derivatised via terminal alcohol group to give ether link which can be coupled to treatment chemicals e.g. scale inhibitor polymer or to create a monomer for copolymerisation. Histidine: amine or acid can be derivatised allowing coupling to treatment chemical e.g. scale inhibitor polymer or to create monomer.

Other methods to incorporate new labels onto polymer backbone (i.e. in a copolymerisation approach) include the use of ‘click-chemistry’ to connect detectable label to polymer backbone. Click chemistry is a well documented way of joining two molecules together and has been used in the synthesis of functionalised polymers (Angew. Chem. Int. ed., 2002, 41, 2596-2599). One example of a highly efficient “click reaction” is the Azide-Alkyne Huisgen Cycloaddition. In this reaction, one molecule is functionalised with an azide group and the second an alkyne group, the reaction is Cu(I) catalyzed and results in the joining of the two molecules through a triazole linkage. This reaction is compatible with a range of functional groups (e.g. alcohols, carboxylic acids, amines) and solvent systems, including water.

For carbohydrates such as galactose, glucose and mannose, a number of possible methods exist. There are a number of literature examples of functionalising carbohydrates with both azide and alkyne groups for use in the click reaction. References below describe the preparation of galactose alkyne (J. Am. Chem. Soc., 1995, 117, page 5395 describes preparation of C-allyl galactoside and Chem. Commun, 2006, page 2379) describes the remaining steps to produce the C-propynyl galactoside and the functionalising a polymer with mannose using the click reaction (Example of synthesising a carbohydrate functionalised polymer: J. Am. Chem. Soc., 2007, 129, 15156-15163).

Hypoxanthine can be derivatised at the 8 and 9 positions with an alkyl chain are described in international patent WO9931104 and U.S. Pat. No. 6,849,735 respectively.

In order to attach cholic acid to a treatment substance, an alkene functionalised cholic acid monomer is described in H. C. Kolb, M. G. Finn and K. B. Sharpless, Angew. Chem., Int. Ed., 2001, 40, 2004

Many biomacromolecules act as part of complexes, with recognition sites for specific small molecules that influence binding and function of the biomacromolecule. Indeed, one of the most common ways in which a molecule may exert its effect in a plant or animal is through a specific interaction with another molecule, the association leading to a cascade of such molecular signalling events. Such a biomacromolecule-small molecule complex is known as a molecular signalling complex. The binding of a target small molecule to its recognition site in the biomacromolecule may lead to displacement of another small molecule, production of a molecule or a conformational, light or colour change in a sample which can be detected. By detecting the displaced molecule, the quantity of the target small molecule present can be determined. Similarly, the emitted light, produced molecule or colour change can be calibrated to the amount of the small molecule that is bound to the recognition site. Such a method is frequently used within the context of biological, biomedical and biochemical fields of application.

In particular, biotin (Formula: C₁₀H₁₆N₂O₃S), also known as vitamin H or B₇, is a good example of a useful label. It is small, commercially available in large quantities and there are a number of functionalised versions available e.g. biotin ethylene diamine, biotin cadaverine and biotin hydrazide which have amine groups that can be used to bind to carboxylic acid-containing chemicals e.g. some scale inhibitors. Biotin is a prosthetic group found on only a few protein species (Ann N.Y. Acad. Sci 447:1-441, Dakshinamurti and Bhagavan, Eds. (1985)). In nature, biotin has roles in the catalysis of essential metabolic reactions to synthesise fatty acids, in gluconeogenesis and to metabolise leucine. One of the most important features of biotin is its very strong binding to streptavidin, avidin, neutravidin and captavidin proteins. Binding of biotin to avidin has a dissociation constant K_{d} in the order of 10⁻¹⁵ mol/L (Bonjour, 1977; Green 1975; and Roth, 1985). This allows for very low limits of detection when using biotin-avidin detection systems. Harsh conditions are required to break the biotin-streptavidin bond i.e. high temperatures, extremes of pH and denaturing conditions.

This strong association has lead to much research into how molecules bind. The strong bond also accounts for the use of biotin is used in many biological applications. For example, biotin may be linked to a molecule of interest for biochemical assays e.g. proteins, amino acids, enzymes, peptides, oligosaccharides and lipids. If avidin/streptavidin/neutravidin/captavidin are added to the mixture then they will bind to the biotin. This can allow capture of the biotin labeled material. Such an approach is typically used in, for example, enzyme-linked immunosorbant assays (ELISA), a biochemical technique used mainly in immunology to detect the presence of an antibody or an antigen in a sample; enzyme-linked immunosorbent spot (ELISPOT), a common method for monitoring immune responses in humans and animals, and affinity chromatography, a method for separating biochemical mixtures (also may be used in protein purification). Application of biotin has been limited to tools for microbiology, biochemistry and medical science. There are no examples of biotin being used to monitor the flow of fluids in systems, or the movement of chemicals and organisms following chemical conjugation to these entities.

However, biomacromolecules themselves are highly sensitive to their surroundings. For example high or low temperatures and solutions of high or low pH can often denature proteins, destroying their ability to bind and affecting their functionality. As a result, amino acid derivatives such as polypeptides are not ideally suited for introduction into fluid conducting and containing systems, either attached to oil or water treatment substances or as free moieties. In addition, biomacromolecules are large, and therefore can have a major impact on the system being investigated.

In another example, equal mM amounts of a model scale inhibitor polyacrylic acid (PAA) polymer is reacted with biotin ethylene diamine, in the presence of EDC as coupling reagent, N,N-Diisopropylethylamine (DIPEA) as base and Dimethylformamide (DMF) as solvent to form an amide bond between the PAA and biotin ethylene diamine. The reaction is monitored by (thin-layer chromatography) TLC to such point that all the biotin ethylene diamine is used up in the reaction. The DMF is then removed by evaporation and the product re-dissolved in methanol. The biotin ethylene diamine PAA product is crashed out of solution using dichloromethane, vacuum filtered and dried to afford an off-white solid.

The ratio of biotin to PAA was estimated by integrating the NMR signals. For this the HOOC—C(CH₃)₂—CH₂ peak of the PAA (1.029 ppm) and the CH-peaks of the biotin (4.13 and 4.30 ppm) were compared. This gave a 1:1 ratio of PAA to biotin, see FIG. 2B.

The biotin labeled PAA can be detected following addition of a second reagent. Preferably, the Biotective assay (FluoroReporter, Invitrogen) was used to detect the concentration of biotin-PAA in samples. It was used according to the manufacturer's instructions. Fluorescence was observed from labeled material but minimal fluorescence observed from unlabeled material. Increasing fluorescence was observed with increasing labeled polymer concentration (FIG. 2C).

Polymeric treatment substances can also be labelled at one or both of the terminal ends of the polymer molecule. The first method involves the reaction of a suitably functionalised label with a suitably end-functionalised polymer also know as a telechelic polymer. Telechelic polymers are well know in the art and are considered to contain one or more reactive end-group (s), which can undergo chemical reactivity with itself or another functional group in another molecule. An important consideration in the use of telechelic polymers is their average functionality, i.e., the average functionality of a monotelechelic polymer should be 1.0 and that of a ditelechelic polymer should be 2.0. Typically end-group attaching reactions are highly sensitive to accurate end group stoichiometry. A wide variety of polymers with reactive end-groups that are different to those of the main polymer have been synthesised. For example U.S. Pat. Nos. 5,393,843, 5,405,911, 4,518,753, and WO9633223 describe the formation of telechelic polymers through the use of organo-alkali metal initiators with subsequent reaction of said polymers, containing active alkali metal end groups, with a reagent which, will either couple the polymer molecules or replace the alkali metal with more stable reactive end groups such as hydroxyl, carboxyl, epoxy, or amine groups. WO2005085297 describes telechelic polymers having a reactive borane residue at one end of the polymer segment resulting from the use of a cycloborane initiator. The borane residue can be converted to at least two functional groups such as hydroxyl, amino, aldehyde, anhydride, halogen, carboxylic acid, etc. WO1996021683 describes combining a living polymer or a polymer having a terminal halide with an alkylsilylpseudohalide. In the invention the alkylpseudohalide contains at least one alkyl group, at least one pseudohalide and at least one Si atom, where the Si atom is bound to the pseudohalide for example an azide, isocyanate, thiocyanate, isothiocyanate or a cyanide. Once the living polymer has been reacted with the pseudohalide, it may be modified to form another functional group by known chemistries. WO2005077987 describes a process for end-capping a cationically polymerized polymer with an anionic group, after which the resulting anionically terminated polymer can be used in subsequent anionic reactions, including anionic coupling and polymerization reactions.

Once polymers with reactive end-groups have been synthesised the polymer can be labeled with non-reactive functional end groups such as the molecular labels or labels described in this patent. For example EP0785422A1 describes polymers having reactive amine thiol end groups which can be used to attach a detectable label (label). The amine thiol groups are imparted through the use of amine thiol based chain transfer agents. The amine groups at the terminal end of the polymer are used during the analysis stage to attach an amine reactive detectable label. The detectable label is only attached to an amount of polymer that has been sampled for analysis. The bulk amount of chemical is not labelled (labeled). This method is only useful for single streams of treatment chemical in relatively clean systems where the attachment of the label would not be susceptible to side reactions. The following article, Langmuir 2007, 23, 8452-8459, describes the synthesis of end-labelled PAA for the purpose of investigating polyelectrolyte systems. Primary amine end functionalised PAA was synthesised via an atom transfer radical polymerisation (ATRP) method. Reaction of a suitably functionalised rhodamine fluorophore with the terminal amine group afforded the end labelled polymer.

The afore mentioned method is a two step process, synthesis of a polymer with reactive end groups followed by conjugation of a label(s) to the polymer specifically at the end group(s). However, labels can be attached to the ends of polymers during the polymerisation reaction either as a moiety attached to an initiator species, transfer species or a end-capping agent species.

The label may also be attached to a polymer by using an initiator species to which the label is already chemically bonded. For example, biotin functionalised ATRP initiators have been described in journal articles such as Biomacromolecules, 2006, 7, 2297 and J. Mater. Chem. 2007, 17, 4015. In these particular articles the biotin is not used as a label to detect the polymer but as a ligand whose purpose is to attach the synthetic polymer to steptavidin protein coated surfaces. J. Amer. Chem. Soc. 2002, 124, 7258 describes the use of a biotin derivatised aryl amine based initiator used in the cyanoxyl mediated polymerisation of a glycopolymer. Conversion of the aryl amine to an arene diazonium cation and reaction with sodium cyanate provided the initiator system for the free radical polymerisation. Another example from U.S. Pat. No. 4,188,478 describes the use of an initiator species that has to it attached a chelating group separated by a spacer. The spacer acts to dissipate any of the inductive effects the end group may have on the initiator part of the molecule, which affects how well it initiates the polymerisation. The patent also describes the use of similar species as terminating agents.

Functionalised transfer agents can also be used to incorporate labels to the ends of polymers. For example biotin has been incorporated to the α-end of a glycoprotein synthesized via RAFT using a biotin functionalised chain transfer agent ref: Macromol. Rapid. Commun. 2008, 29, 511-519. A bipyridine functionalised RAFT transfer agent described in J. Polymer Sci: Part A: 2007, 45, 4225-4239 was used to produce bipyridine end-functionalisd polymers. J. App. Polymer Science, 2004, 91, 2035 describes how different groups including a dodecane group were attached to the end of polyacrylic acid using thiol based chain transfer agents.

The synthesis of polymers with phosphonate end groups is described in U.S. Pat. No. 6,071,434A1. These phosphate end caps are used to provide additional biodegradation and adorption properties to scale inhibitors. These polymers are similar to block co-polymers in that the phosphonate entities are polymerised into a short terpolymer from which the rest of the polymer is then grown.

While the following references do not specifically relate to the co-terminal attachment of labels or labels to polymers they do describe how end-capping or end-labelling of polymers with non-reactive groups may be achieved. U.S. Pat. No. 5,015,692 describes polymer end functionalisation through terminating reactions of alkali metal containing polymers with nitro compounds, phosphoryl chloride compounds, and amino silane compounds with attached alkyl or alkoxy groups. As well as methods for incorporating the above functional groups U.S. Pat. No. 5,128,416 also includes methods for the end-functionalization with acrylamides, and aminovinyl silane compounds in combination with conventional silicon or tin coupling compounds. WO8703603 describes how Ziegler-Natta catalyzed polymer chains are end capped with at least one functional group-containing unit which is otherwise essentially absent from said polymer chain. As part of the afore-mentioned the process comprises end capping the polymerization with vinyl pyridine is described. The patent also mentions that functional groups from the group consisting of: isocyanates, urethanes, nitriles, aromatic ethers and aromatic carbonates may also be used.

The method described thus far relates to the addition of the tag, functionalised with either a end-capping agent or an initiator of polymerisation, to monomer units of the polymeric treatment substance so that the polymer is tagged during synthesis. However, another method of terminally tagging a polymeric treatment substance is to attach the tag post polymerisation, to a polymeric treatment substance. In such a case, at least one monomer of a polymeric treatment substance is polymerised prior to conjugation of the tag.

For the polymerisation, it is preferable that a polymerisation initiator is used, the polymerisation initiator having a functional group that is capable of reaction with the detectable tag. It is also preferable that a polymerisation end-capping agent is used, the polymerisation end-capping agent having a functional group that is capable of reaction with the detectable tag. This method is advantageous because the functional group is added a terminal end. When the functional group reacts with the tag, therefore, the polymeric treatment substance will have a single detectable tag.

It should be noted that a functionalised initiator or end-capping agent would be used, so that only one end of the polymeric treatment substance is tagged. If it is desired that both ends of the polymer should be tagged, then a polymerisation initiator and an end-capping agent could be used, both the initiator and the end-capping agent having a functional group, the functional group being capable of reaction with a detectable tag. This would allow increased sensitivity of detection, where a tag is particularly difficult to detect but otherwise desirable to use for example it is cheap or non-toxic. It would also offer the possibility, where the functionality of the end-capping agent and initiator are different, of attaching two different tags. This could be of particular use where two treatment substances are carrying the same, first, tag; the user could then use a detection molecule selective for the second tag.

The polymerisation initiator may be an atom transfer radical polymerisation initiator and a halogen functional group may be added to a terminal end of the polymeric treatment substance. Atom transfer radical polymerisation initiators carry a halogen function group. Where this is the case, the halogen functional group may be converted to another functional group suitable for reaction with the tag. Non-limiting examples of such groups include a triazole or ether linkage. Atom transfer radical polymerisation reactions are especially easy to control, so that the certainty of attachment of one tag to one polymer molecule is increased.

### Example 2

### Scale Inhibiting Activity of Labeled Scale Inhibitors

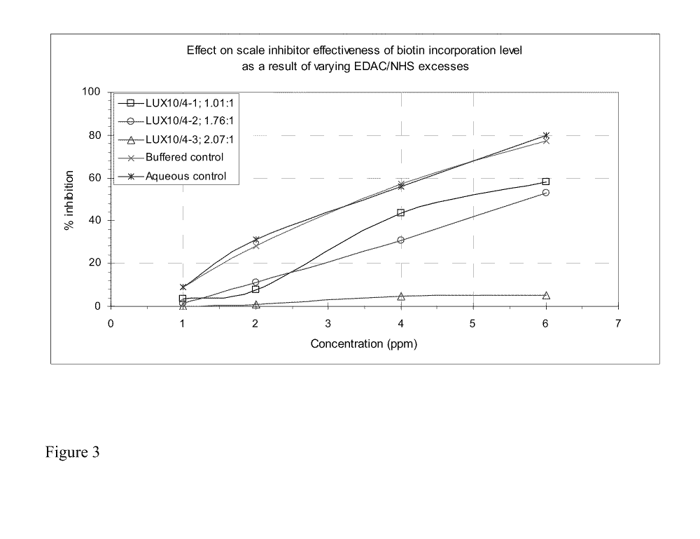

The scale-inhibitor activity of labeled scale inhibitors was determined using static bottle tests (barite). This test is used to assess how efficient the chemicals are at inhibiting scale build-up compared with the unlabeled original chemicals. Inhibitors, labeled or unlabeled, were analysed in duplicate in 50:50 Forties Formation Water: Seawater at 95° C., tested after a 22-hour incubation. Solutions are dosed with inhibitor and incubated. Undosed solutions serve to provide a ‘base-line’ scaling potential of the water system. After incubation, the aliquots are sampled and the concentration of the scaling cations of interest in each sample is determined by ICP-OES (inductively coupled plasma-optical emission spectrometer). This analysis method is known by the one skilled in the art of detecting, identifying and/or quantifying single chemical elements. Results from one such test are shown in FIG. 3. They indicate that decreasing scale inhibition activity is caused by increasing incorporation level, with a 1:1 incorporation causing some, but relatively small decrease in the capacity of the inhibitor-to-inhibitor scale formation.

### Example 3

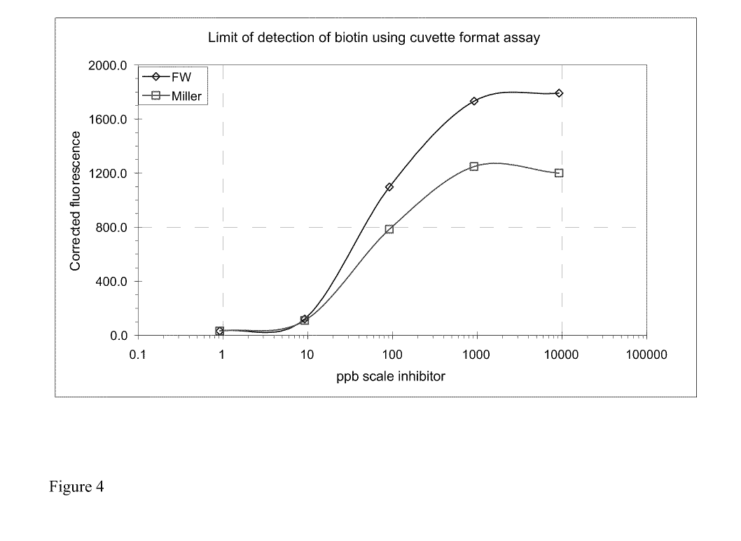

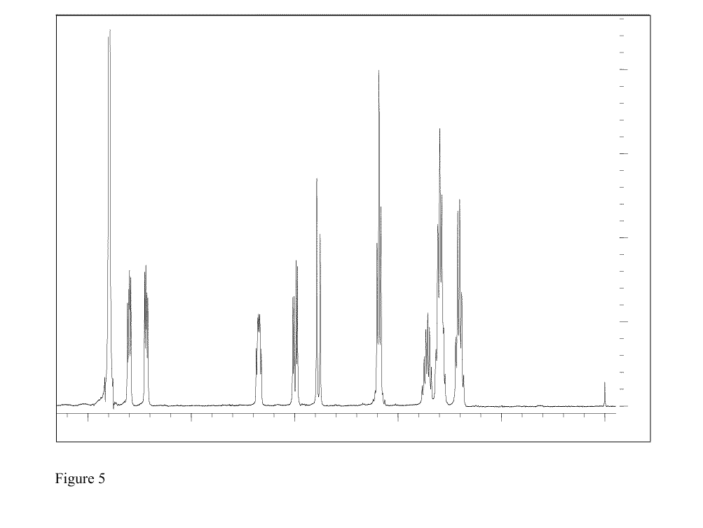

### Limits of Detection of an Exemplary Labelling Molecule

Where the label in question is added during production of the treatment substance, for example during copolymerisation of polymeric scale inhibitors, more label molecules may be incorporated if it is desired to increase the detectability of the conjugate on addition of the associated biomacromolecule. Conversely, if the signal created on addition of the biomacromolecule is excessive and difficult to measure, the amount of label may be reduced. The limits of detection of the labels range from a concentration of 1 part per billion to parts per million. For treatment substance-label conjugates to be useful they need to be able to be detected at very low levels. Continuously injected scale inhibitors are typically loaded into the wells at 5-500 ppm. For squeeze treatments, the inhibitors may be resqueezed when the inhibitor reaches 1 ppm. Therefore, the limit of detection of modified treatment substances will ideally be below 1 ppm. Biotin-labeled scale inhibitor (1:1 ratio biotin:polymer) was added to the aqueous phase of produced fluids obtained from the Miller Field. A modified protocol of the Biotective Green assay, which utilized larger volumes of reagent in a cuvette format and the PicoFluor fluorometer (TurnerBiosystems) was used to determine the concentration of biotin present, and thus the concentration of scale inhibitor, see FIG. 4. Results indicate that limits of detection to 20 ppb treatment substance are possible.

### Example 4

### Verification of Labelling and Detection of Scale Inhibitors with Biotin

The method of maintaining correct function of a fluid conducting and containment system as described hereinabove can be used to identify, for example, leakage, pipe corrosion or build up of scale or hydrate and this information used to inform the design of a subsequent treatment schedule. The flow of fluid in oil wells, or flow of water in a river system, for example identification of source, location of fluid, concentration of particular components, time of flight, contribution of different wells or rivers and partitioning characteristics may be assessed in addition to the monitoring of treatment compositions using this method. This method may be automated.

Further experiments were performed to confirm that polymeric scale inhibitor which had been labeled with a latently detectable label could be detected in an industrial setting. The scale inhibitor used contains phosphorus, unlike the biotin marker or buffers used. Inductive Coupled Plasma Spectroscopy can therefore be used to assess the concentration of inhibitor in final solution of labeled chemical. Calibration of the equipment using known concentrations (5, 10 and 50 ppm activity inhibitor in 1% Na⁺ brine) of unlabeled inhibitor allowed quantification of inhibitor in the sample to be determined. The method used low wavelength of 177.440 nm (Plow) in Gaussian mode. This uses 7 points, calculates on 3, has a 5 sec integration time and uses narrow slits 18 and 15.

The data from the concentration of scale inhibitor was compared to that from concentration of biotin and used to estimate the ratio of biotin:polymer in any given sample. NMR and size exclusion was used to verification of the labelling of biotin to the inhibitor.

FIGS. 4 and 5 show NMR verification of the labelging of biotin to inhibitors. This technique uses radiofrequency pulses to manipulate the quantum spin of individual atomic nuclei. The resulting spectrum consists of a peak for each non-identical nucleus. In these experiments only hydrogen nuclei (proton NMR) were measured. The sample was recorded with 64 scans and water pre-saturation on a Bruker AV600 spectrometer. Samples were prepared by adding methanol to the solutions, which result in precipitation of the polymeric species. The solid was dried and dissolved in D₂O for analysis. A spectrum (400 MHz) for biotin, FIG. 4, is freely available via: http://bmrb.protein.osaka.ac.jp/metabolomics/gen_metab_summary_{—}5.php?m of Name=biotin. Labeled inhibitor was analysed for the presence of these peaks that would be diagnostic of biotin presence. The spectra are shown in FIG. 5 and showed the presence of biotin peaks as well as the broad polymer peaks on the baseline. The biotin peaks show broadening relative to the reference spectrum suggesting an increase in molecular size and is therefore further evidence of coupling.

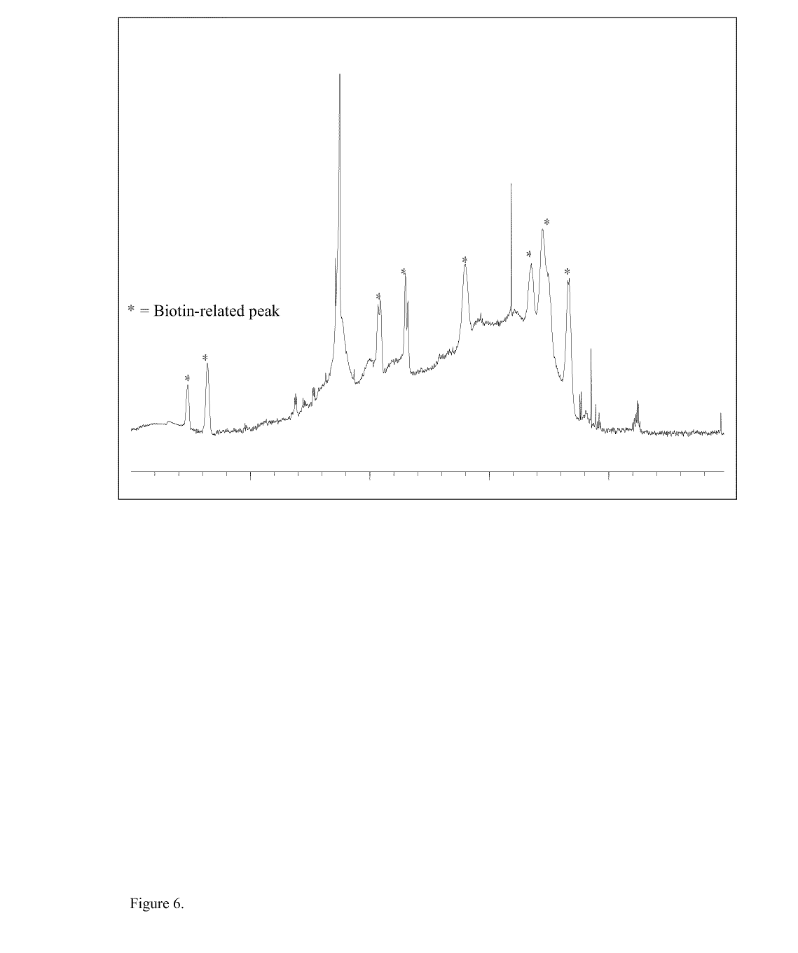

Size exclusion chromatography (sometimes called gel permeation or gel filtration chromatography) was used to separate constituent components by size. The rationale was that if biotin is not labeled to the chemical it will be observed as an increased population of smaller-sized fractions. A chromatograph of starting materials for reference is shown in FIG. 6. The chromatograms indicate that the biotin and biotin ethylenediamine peaks were slightly shifted relative to each other, which demonstrates the resolving power of the column at these low (300-400 Da) molecular sizes. The peaks at ˜11 mins are the inorganic ions. The scale inhibitor polymer (chemical 2) chromatogram shows a broad distribution of polymer peaks. The labeled chemical chromatograms indicate a small peak present, which appears to correspond to biotin ethylenediamine and this may be due to some starting material that was not removed during purification. Further, there is a shift in the maxima of the polymer peaks of the conjugates relative to the starting polymer, which indicates that coupling has successfully occurred.

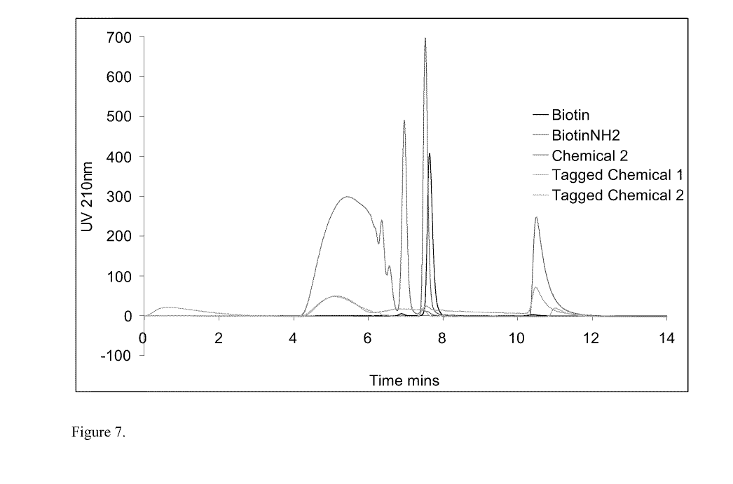

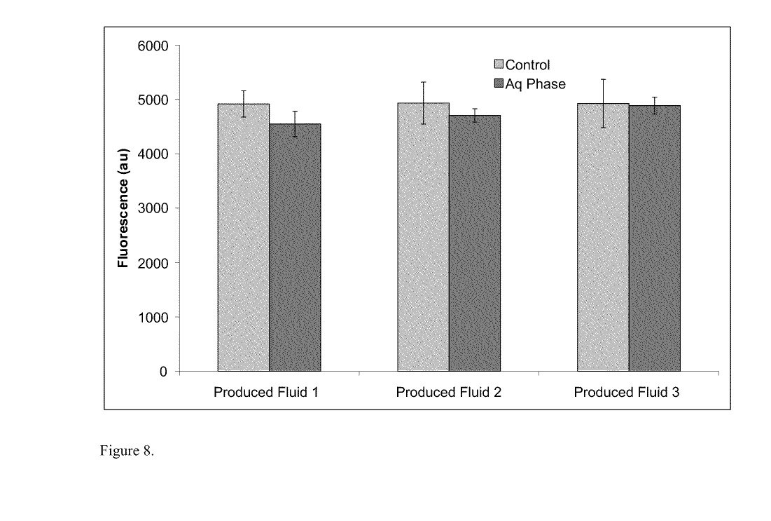

Scale inhibitors are known to partition into the aqueous phase. To be effective, labeled chemicals should show similar partitioning behaviour to unlabeled chemical. In the experiments labeled scale inhibitor chemical was added to various produced fluids (mix of oil and aqueous phases). The solutions were mixed well and left to shake overnight at room temperature. The amount of labeled chemical in the aqueous phase was then determined and compared with control samples (no oil phase), see FIG. 7.

Given the data, we are confident that biotin-labeled scale inhibitors will partition to the aqueous phase and therefore they will be effective in fluid conducting and containment systems.

The label may be conjugated to at least one treatment substance instead of being used as a free label. The labelled treatment substance could constitute 100% of the treatment substance, or a proportion of it, so that a mixture of labelled and unlabelled treatment substance may be used. The labelled treatment substance may then be added to a system, a sample taken from the system and the associated biomacromolecule added to the sample. The biomacromolecule should be added in a predetermined quantity sufficient to interact with the label in order to cause a measurable change in the sample, and the change in signal measured and analysed. For example, a standard curve of the signal emitted from solutions of different concentrations of the labeled treatment substance, when the biomacromolecule is added, could be used to determine the concentration of treatment substance in the sample. In this case the concentration of biomacromolecule added to the sample must be the same as that used to prepare the standard curve. The depletion of one or more particular treatment substances during operation of the fluid conducting and containment system could then be detected due to a reduction, increase or other change in the signal emitted from the sample. In this case, the data obtained is used to inform the administration of at least one treatment substance into the fluid conducting and containment system in order to maintain minimum inhibitory concentrations (MIC) of said compounds. The method may also, therefore, involve the administration of treatment chemicals into the system, a mechanical adjustment or any other necessary action. This process may also be automated

Multiple labels can be used in a fluid conducting and containment system to improve monitoring. Labels could be differentiated according to the type of spectra they emit, by the method used to detect them such as fluorescence, luminescence or colourimetry, or the labels may have different fluorescence lifetimes. Combinations of labels which can be differentiated in this way may be used advantageously, for example, to analyse the distribution of different treatment chemicals or different components of a treatment formulation, to determine the contribution that different wells or rivers may make to production, to assess mixing of fluids or components of fluids or to monitor different microbes. This process may also be automated. Preferably, multiple labels are used in subsea oil fields where, because of subsea completions flow from several individual wells becomes combined and piped to the nearest platform. In such a case it is difficult to determine the well wherein inhibitor should be added, unless the inhibitor or inhibitors, should they be different, used in each well are reacted to a different label.

The label may also be conjugated to microorganisms or to nutritional elements taken up by microbes to allow monitoring of the movement of organisms in pipelines, the sea, rivers or canals. Such microorganisms could include bacteria, viruses, fungi, protozoa, algae, plants and algae. The labels may be coupled to the organism using amine chemistries, antibodies, antibody fragments, aptamers or molecular imprinted polymers.

At a point of monitoring, the fluid may be analysed by in-line or on-line techniques by adding the associated biomacromolecule to the system and incorporating a detector into the system. Alternatively the sample may be removed from the fluid conducting and containing system either as part of a batch process or continuously. In other words, individual samples may be taken from the fluid in the system and tested at pre-determined timepoints, or alternatively the fluid can be continuously sampled, for example by having a diversion from a main flow pipe. The sample may be processed to enhance detectability of the treatment substance or to isolate a particular fraction, for example, that containing material of a certain molecular weight such as the small molecule alone, or labeled chemical. This process may involve, for example, immobilisation of the label, size exclusion chromatography, centrifugation, ultrafiltration, tangential filtration. Alternative processing methods could include chromatographic methods such as hydrophobic interaction, reversed phase. Such a process may also serve to concentrate the sample, so increasing sensitivity and in turn reducing the amount of small molecule which may be required. It may also remove any interfering compounds e.g. algae which may autofluoresce in a similar region to labels/detection technology. If using a filtration process the retentate would be kept and if using size exclusion the desired fraction would be kept. The final sample size is process dependent and as the person skilled in the art would know, it would vary between the range 1 microlite to 1 litre. Further, the physical characteristics of the sample may be altered to improve sensitivitity/quantification by adding appropriate reagents known to the person skilled in the art.

The person skilled in the art would understand that the method of detection will vary depending on the label attached to the treatment substance, the associated protein that interacts with it, and the type of signal emitted as a result of the interaction. The label-protein complex may be detected directly using vibrational spectroscopies such as Raman or infrared (IR) spectroscopy. These methods require the use of labels that are active; IR selection rules require that the vibrations induce a change in symmetry whereas Raman selection rules require an alteration in polarisation.

Raman is the inelastic scattering of light. It is based on the process of Stokes or anti-Stokes Raman scattering, generated as an electron in a molecular bond moves back to its orignal electronic state but different vibrational state after the molecular bond is exposed to incident light of an appropriate wavelength. A sample is illuminated with a laser beam, light from the illuminated spot is collected and the Rayleigh scatter is filtered to leave behind the Raman scatter. Usually, a photon-counting photomultiplier tube (PMT) or CCD camera is used to detect the Raman scattered light. The spectra determined by Raman spectroscopy provides a ‘fingerprint’ of the sample enabling determination of its components, such as the presence of the small molecule-protein complex in question. In contrast to Raman, which is the scattering of light, IR is based on the absorption of light, and can show different, complementary information. IR spectroscopy shows transmittance of infrared light through a sample and is typically observed as inverted peaks due to vibrational absorption. Other suitable spectroscopy methods are known to those skilled in the art.

In another detection method, the labels are detected following addition of a reagent that contains the associated protein component via an emitted signal. As the protein and label interact, a detectable signal, usually a light, temperature or colour change, is generated and measured. The protein is added in excess to ensure complete binding of the label to the protein occurs.

To generate a signal the proteins themselves may be modified, for example with a fluorophore (with dye, protein, or quantum dot), luciferase or peroxidase. Patent WO2005080989 refers to an example of a biotin recognition compound (BRC) in which fluorescence is produced on binding of the biotin to a modified biotin-binding protein. The binding of biotin to this protein removes a quencher allowing fluorescence to be generated from a fluorophore attached to the streptavidin protein. The intensity of the fluorescence is related to the amount of biotin present.

Where a signal is not generated directly on addition of the associated protein it may be possible to use a ‘competition’ assay to determine the concentration of the label in the solution. A binding event between the label on the treatment substance or microbe and associated protein is still exploited in this method. The sample containing the label is placed in contact with a surface coated in the associated protein. The label in the sample binds to the associated protein on the surface of a vial, microplate, or bead (protein in excess). The surface is washed to remove unbound label. A detectably labelled molecule is added to the surface. The detectable label may be a fluorescent protein, luciferase, dye or Quantum dot. This labelled molecule binds to any remaining unbound protein on the surface. Where more labelled treatment substance or microbe bound initially there will be fewer available binding sites for the labelled small molecule and less signal will be obtained. Therefore, where a higher concentration of labelled treatment substance or microbe is present in the sample, a reduced signal will be detected. Protein-coated surfaces are commercially available. For example, streptavidin-coated vials and microwell plates. Labelled small molecules are available e.g. biotinylated luciferases (Avidity) and fluorophore labelled biotin (Fluorescein biotin, product number B1370, Invitrogen)

In a second competition assay that may be used to detect the labels of the present invention, an associated protein, modified with an identifiable marker such as a fluorophore or luciferase, is added in excess to the sample containing the label. Unbound protein is removed e.g. using size exclusion, with magnetic beads coated in the small molecule, or by flowing the solution across a surface coated with the small molecule. The amount of the label-associated protein complex remaining in the solution is measured by determining the amount of identifiable marker. This marker may be fluorescent e.g. protein, dye or quantum dot, or luminescent. For the latter substrate is added e.g. D-luciferin and ATP and light generated. Fluorophore labelled proteins are commercially available (e.g. streptavidin fluorescein conjugate, catalogue number S869, Invitrogen). Luciferase-conjugated proteins are available (Nakamura M., Mie M., Funabashi H. and Kobatake E. (2004) Construction of streptavidin-luciferase fusion protein for ATP sensing with fixed form. Biotechnology Letters 26 (13) 1061-1066).

The reagent(s) used during detection depends on the application, method of detection and volumes used. As the person skilled in the art would understand, these aspects of the detection method will need to be optimised. The application will affect the amount of sample tested and reagent used. For example, monitoring of water movement in a river system may require larger sample volumes and so more reagent than a water-cooling tower. The volume of reagent required is expected to range from microlitres for low volume systems to litres where a flow-through monitoring system is used. The concentrations of reagent used depend on the method of detection used. For example, 4 biotin molecules are bound by one avidin molecule. However, different forms of avidin are available. For example, a monomeric avidin protein has been developed that binds only one biotin molecule [Laitinen O. H., Marttila A. T., Airenne K. J., Kulik T., Livnah O., Bayer E. A., Wilchek M., Kulomaa M. S. (2001). Biotin induces tetramerization of a recombinant monomeric avidin. A model for protein-protein interactions. Journal of Biological Chemistry 16; 276(10:8219-24]. Therefore, the use of different small molecules and proteins will influence the concentration used.

The reagent added may also contain other components to optimize generation of the light signal. For example, it may be used to buffer the sample pH to that at which optimal light is produced. The optimal pH depends on the marker used; between 2 and 4 for rhodamine, pH 7 for fluorescein. If luciferases are used, the pH and salt content of the media may be optimized by addition of suitable reagent, thus Gaussia luciferase works optimally at pH 7.8 and in 500 mM sodium chloride. Firefly luciferase is ATP dependent and this would need to be added in the reagent if this protein was used. These modifications are routine and the person skilled in the art would easily understand the adjustments required.

The apparatus used to measure the signal generated as a result of the interaction between the label and the associated protein will be selected by the person skilled in the art, depending on the type of signal generated. The limit of detection of the labels according to the invention ranges from parts per billion to parts per million, depending on which label is used.

Where the signal generated is a colour change, visible light absorbance may be measured, for example with a spectrophotometer. Where the signal generated is light, equipment is required which can measure light. This may be a luminometer (plate readers, tube luminometers, portable cuvette-based luminometers), fluorometer, (plate readers, tube fluorometers, portable cuvette-based fluorometers), CCD camera, photon counter, photographic film, photometric detector, Raman spectrophotometer, Infrared spectrophotometer, MEMS device, chromatography system or the signal may be perceived visually by eye. Many devices are commercially available. These may be bench top or portable devices. Further, adaptors are available to ease detection, for example, using fibre-optic bundles to determine light production at a distance from the detector.

The use of a label that is only detectable in the presence of an associated biomacromolecule is advantageous for a number of reasons. The interaction between an associated biomacromolecule and the molecule with which it interacts is extremely specific, because the biomacromolecule such as protein has a molecular recognition site into which only the label will interact. Therefore, the user can be certain that any change in signal detected on addition of the associated protein is due to the presence of the label. Another advantage becomes apparent where solutions such as oil, treatment chemicals and water obtained from the environment, which have significant fluorescence, are to be tested. The use of a fluorescent moiety, which is a common choice of label, can therefore create problems in signal processing due to autofluorescence from the sample. The treatment composition of the invention, on the other hand, can be conjugated to a label that can be detected with luminescence, colour changes, Raman spectroscopy or any other non-fluorescent method to avoid background noise. Alternatively, the user may first determine the autofluorescence of the sample, and then add the protein that allows detection of the label. Even in the case that the protein fluoresces in the chosen wavelength, the ‘background’ fluorescence may be subtracted from the signal obtained after addition of the protein. Alternatively, the user can reduce autofluorescence, for example but not limited to adding in a photobleaching step.

### Example 6

### Experiments to Show Resistance of Label to Conditions Typical of Industrial Fluid Systems

Proteins are highly sensitive to their surroundings, for example high temperatures and solutions of low pH can often denature proteins, destroying binding and function. As a result, amino acid derivatives such as polypeptides are not ideally suited to being attached to oil or water treatment chemicals. In contrast, the small organic molecules associated with the protein may be more robust, meaning they can survive in harsher systems. Further, their smaller size should reduce their impact on the system being investigated when compared to the larger proteins.

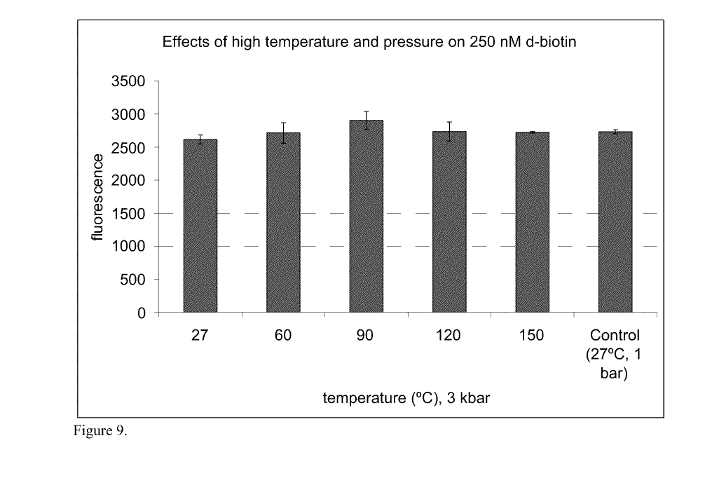

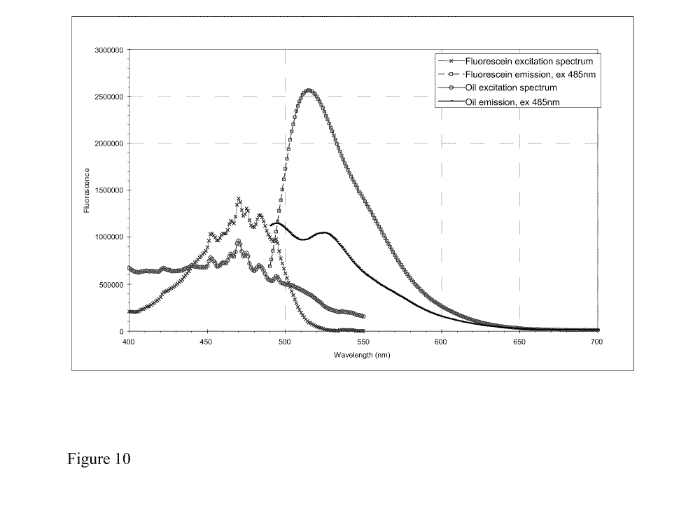

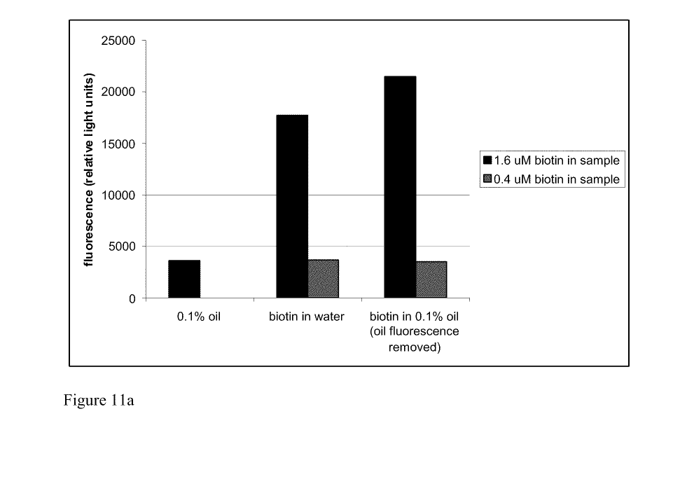

The resistance of d-biotin to high temperatures and pressures was investigated. d-biotin was diluted in formation water and exposed to 15 minutes of 3 kbar pressure at 28, 60, 90, 120 or 150° C. A dilution series was made and the ability of treated and untreated samples to bind streptavidin was determined, using the Biotective Green assay. There was no obvious drop in fluorescence even after exposure to 150° C., 3 kbar, for 15 minutes, FIG. 9. Similar results were obtained when the biotin was heated in the presence of the aqueous phase of produced fluids (FIG. 10); once again, no loss of fluorescence was detected due to temperature, even at 150° C. Biotin appears sufficiently robust to high temperatures and pressures to be used as a label.

### Example 7

### Data Showing the Advantage of Using Latently Detectable Labels and Impact of Background Interferences

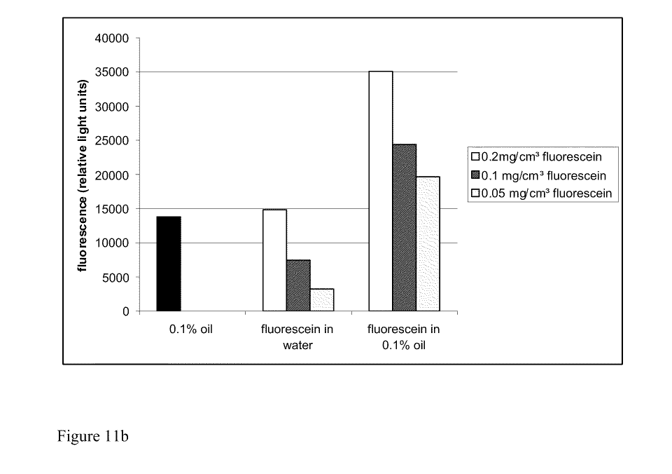

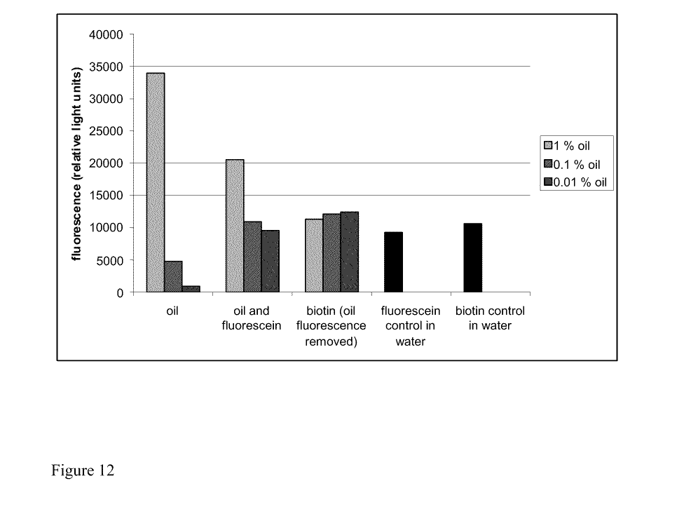

Many fluids in containment systems interfere with the detection of labels. Fluids may be coloured, or have autofluorescence, such as oil solutions. Where the label is fluorescent it will be difficult to quantify the amount present if there is interfering autofluorescence from the sample. However, if the label is latently detectable then the autofluorescence from the sample can first be assessed, then the fluorescence directly attributed to the label determined. This is the case in FIGS. 11a and 11b and FIG. 12, where quantification of a latently detectable biotin label in oil is compared with fluorescein, a commonly used fluorescent label.

In both experiments, fluorescence from samples was detected at 485 nm excitation and 535 nm emission. Oil is also known to fluoresce at this excitation and with overlapping emission, see spectra in FIG. 10, which shows excitation and emission spectra of 0.1 mg/cm3 fluorescein and the oil fraction from Miller field produced fluids, diluted to 0.1% in petroleum ether (non-fluorescent). For fluorescein-containing solutions samples were measured directly and for solutions containing latently detectable biotin, fluorescence from the oil solution was first determined at 485/535 nm (excitation/emission) and then Biotective Green assay reagents (Invitrogen) were added to determine fluorescence associated from the biotin, also at 485-535 nm. Measurements were performed in quadruplicate and the average taken.

FIG. 11a shows the level of fluorescence detected using from various concentrations of biotin in deionised water or 0.1% oil (the oil phase of produced fluid from the Miller field). FIG. 11b shows the fluorescence detected from various concentrations of fluorescein in deionised water or 0.1% oil (as above).

FIG. 12 shows the fluorescence detected when 1%, 0.1%, 0.01% of oil (the oil phase of produced fluid from the Miller field) was mixed with one concentration of label, either 0.8 μM biotin or 0.1 mg/cm3 fluorescein. Control samples i.e. those without label were used to quantify oil autofluorescence.

Both fluorescein and biotin-biotective green cause an increase in fluorescence, beyond that from oil. The difference is that for biotin the background oil fluorescence can first be measured then removed providing reliable data for a range of oil and label concentrations. For fluorescein it is important to know beforehand the oil concentration so the end user can determine what fluorescence is from the fluorescein and what is from the oil. In real fluids this concentration may vary and may lead to difficulties in quantification of a directly-fluorescent label.

### Example 8

### Data Showing the Advantage of Using Latently Detectable Labels and Pretreating Samples to Minimise Background Interferences

Where a latently detectable label is used a sample which contains background interference, such as autofluorescence, can be first treated in some way to minimise autofluorescence. This may be achieved in a number of ways such as addition of chemicals, heat treatment or the bleaching of a sample with autofluorescence. The manner of treatment depends on the sample. This is unlikely to be a viable method if a directly fluorescent label is present since these may be adversely affected by the treatment although the latently detectable labels described here are robust and should remain unaffected.

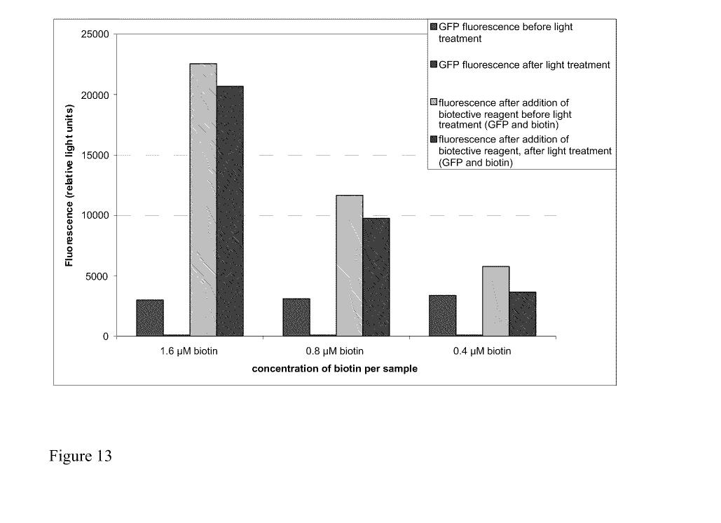

We took a solution of GFP (0.1 mg/ml Renilla reniformis protein, 80%, in water) and added biotin. The sample has high fluorescence from the GFP. This solution was treated in 2 ways (a) no treatment (b) heat treated (samples were heated to 100° C. for 1 hour in an oven). After treatment fluorescence from the sample was assessed, 485/535 nm excitation/emission, both before and after addition of Biotective Green reagent (Invitrogen) which detects biotin. Results indicated that GFP fluorescence was lost after heating, while the biotin was unaffected, FIG. 13.

Latently detectable labels are therefore ideal when samples can be treated to minimise inherent fluorescence or background signal. Since such treatment can adversely affect directly detectable fluorescent labels latently detectable labels have an advantage.

### Example 9

### Limits of Detection of Glucose

The small size and simple structure also make it a good candidate for labelling. A commercially available Amplex® Red glucose assay was used to determine glucose concentration. An Amplex UltraRed® glucose assay could also be used. Glucose oxidase oxidizes D-glucose to D-gluconolactone producing hydrogen peroxide. In the presence of horseradish peroxidase, H₂O₂ reacts stoichiometrically with Amplex® Red to generate the fluorescent product resorufin which can be detected fluorometrically or spectrophotometrically. The effects of high temperatures, low pH, treatment chemicals, various solvents, high salt concentrations, oil and biotin on detectabilty glucose were investigated.

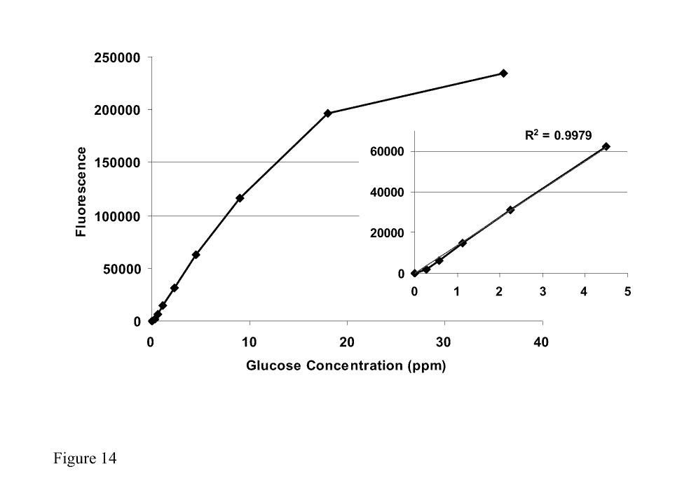

To determine limits of detection of glucose, initially a calibration curve was generated by analysing glucose solutions prepared by serial dilution (36, 18, 9, 4.5, 2.25, 1.125, 0.5625, 0.28125 and 0 ppm). All concentrations quoted refer to the concentration of the solution before addition of the 50 μL of enzymes and reagents for analysis. Results indicate that the glucose calibration curves were relatively reproducible (FIG. 14). The limit of detection was ca. 0.3 ppm.

### Example 10

### Tolerance of Glucose Assay to Synthetic Formation Water

To determine whether the glucose Amplex Red assay could tolerate synthetic formation water, two glucose solutions were prepared by diluting the stock solution (400 mM) to 18 ppm and 3.6 ppm with formation water.

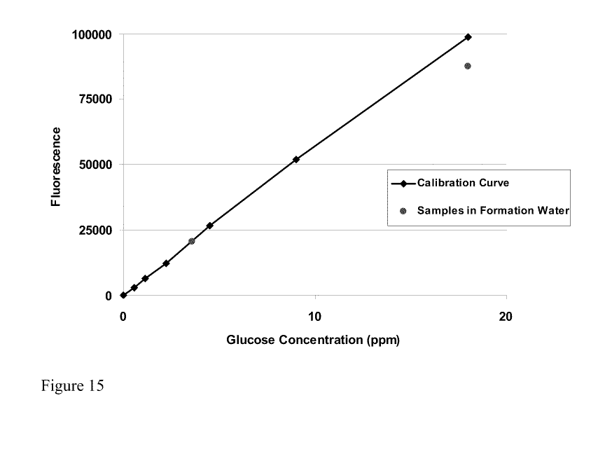

Results indicate that the assay tolerates the presence of formation water (FIG. 15)

### Example 11

### Tolerance of Glucose Assay to Presence of Treatment Substances

To determine whether the glucose assay could tolerate the presence of treatment chemicals, the effects of scale inhibitor, corrosion inhibitor, isopropanol (IPA), methanol and monoethylene glycol were determined A 10% scale inhibitor solution was prepared by adding 100 μL of scale inhibitor 8017 C to 100 μL of glucose (50 mM) and 800 μL of formation water. A 1% solution was prepared by adding 10 μL of scale inhibitor 8017C to 100 μL of glucose (50 mM) and 890 μL of formation water. Controls were prepared by the same method, substituting deionised water for the scale inhibitor. These samples were left at room temperature for 4 h and then serial diluted 1:10 twice, to give a final concentration of 50 μM glucose. 10% and 1% corrosion inhibitor EC1440A solutions were prepared in the same way.

Aqueous solutions of methanol, IPA and MEG (20%) were serial diluted 1:10 with water to give 2% and 0.2% solutions. A stock solution of 100 μM glucose was used. 1 mL glucose solution was added to 1 mL of each concentration of each solvent to give 12 samples with 10%, 1% and 0.1% final solvent concentration and 50 μM final glucose concentration. A control containing 1 mL water added to 1 mL glucose solution was also prepared.

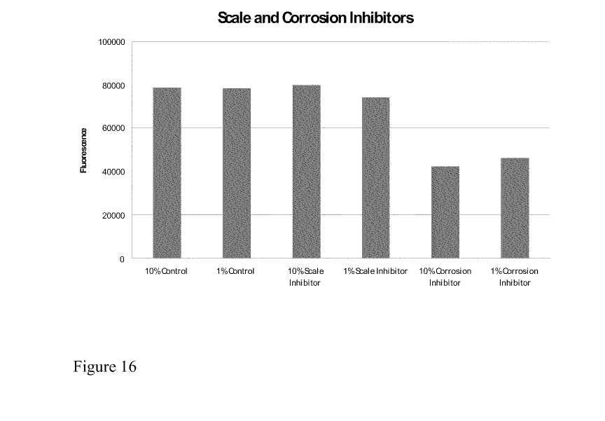

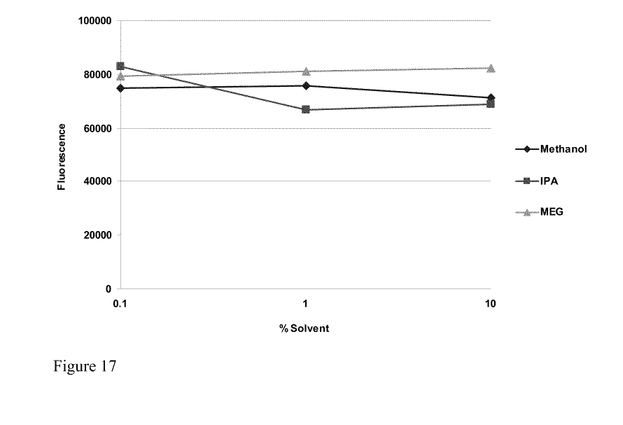

The results can be seen in FIGS. 16 and 17. Scale inhibitor 8017C did not have any effects on the glucose assay. The presence of both 10% and 1% corrosion inhibitor EC1440A markedly decreases the amount of glucose detected although this concentration is well above that expected to be encountered in produced fluids (0.1% is considered a maximum amount expected).

### Example 12

### Tolerance of Glucose Assay to the Presence of Additional Labels

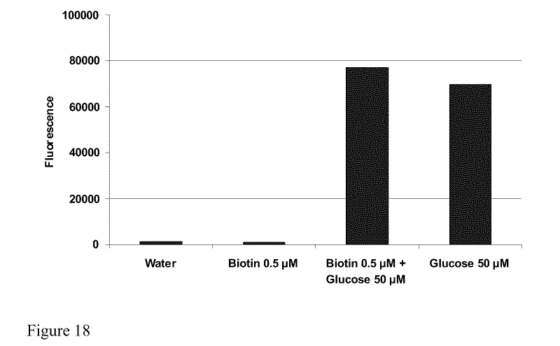

To determine if the glucose assay could function even in the presence of other labels or tracers, so enabling multiple labels to be used at once the effects of inclusion of biotin in the solution was determined. The following four samples were prepared and analysed: 1) Water, 2) Biotin (0.5 μM), 3) Glucose (50 μM), Biotin (0.5 μM) and Glucose (50 μM). Results indicate that the assay tolerates the presence of biotin (FIG. 18).

### Example 13

### Thermal and Acid Stability of Glucose

To determine the thermal and acid stability of glucose, 0.5 mM glucose solutions (10 mL) were prepared in both deionised water and formation water. These solutions were divided and the pH of one water sample and one formation water sample was adjusted to 2 with HCl. A 0.5 mL aliquot was removed from each sample before incubation to prepare control samples. The remaining 4.5 mL were placed in 4 duran bottles with Teflon tape wrapped around the threads to prevent evaporation. After heating at the required temperature (100, 120 or 150° C.) for 20 h, the bottles were cooled to room temperature and diluted 1:10 with deionised water.

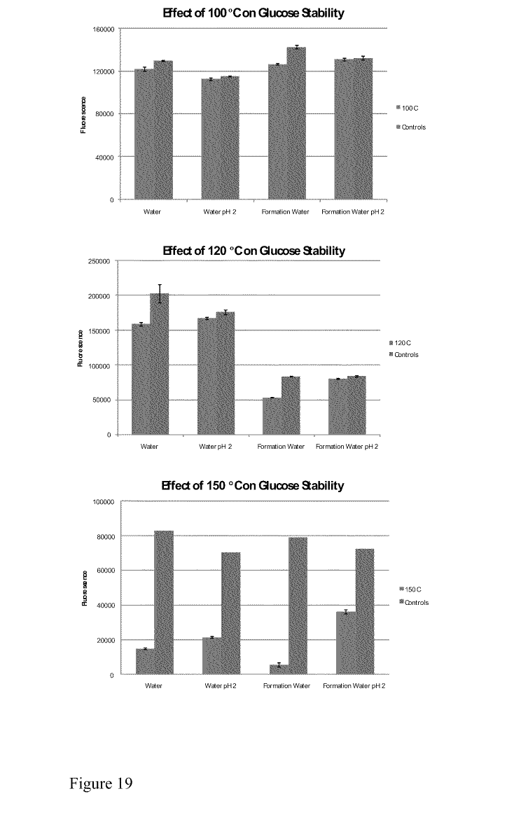

Results are shown in FIG. 19. Samples heated to 100° C. showed no difference in detectability, although at 120° C. there was some evidence of degradation and at 150° C. samples showed a marked decrease in concentration compared to controls. These results indicate that glucose would be best applied to cooler systems, ideally those at or below 100° C. Incubation in solutions of pH 2 did not adversely impact glucose detection.

### Example 14

### Tolerance of the Glucose Assay to Oil

To determine the effects of oil on the assay a 2% oil sample was prepared by adding 2% oil to 98% water by volume. The vial was shaken vigorously by hand and then serial diluted with water to ca. 0.2% and 0.02%. 50 μL of each oil concentration was added to 50 μL glucose solution (100 nM) to give final oil concentrations of 1%, 0.1% and 0.01%. The controls consisted of 50 μL of each oil concentration plus 50 μL water; as well as a 50 μL glucose solution (100 nM) plus 50 μL water sample.

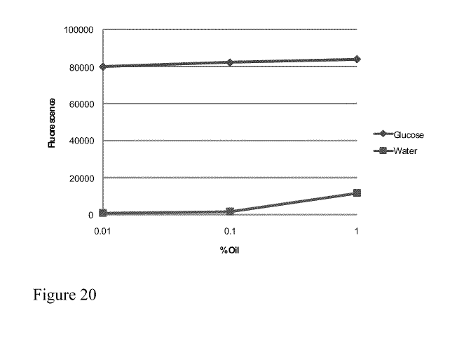

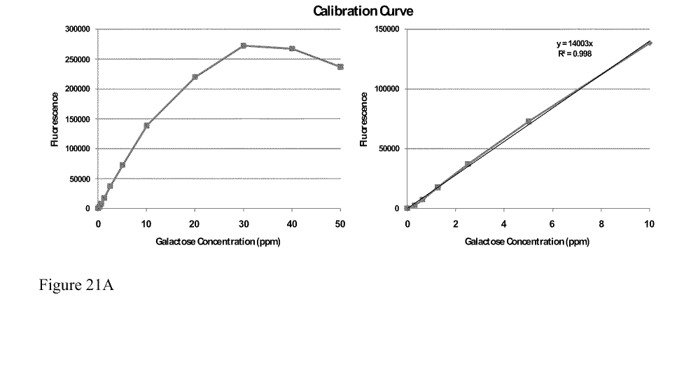

Results (FIG. 20) indicated that as expected low levels of background fluorescence were observed for the oil plus water controls which increased with increasing concentration of oil. The assay, however, appeared unaffected indicating it could be used in oil-containing samples. Again, by first assessing background and then running the assay the latently detectable glucose label or tracer allows interfering background fluorescence to be removed.

Glucose is suitable for labelling treatment substances, and for being detected within the context of an aqueous or organic solution. The limit of detection was ca. 0.3 ppm. The presence of oil, biotin, formation water, methanol, IPA, MEG and scale inhibitors did not have any significant effect on the levels of glucose detected by the assay. Glucose was found to be relatively stable at 100° C. however at 150° C. the concentrations detected were dramatically decreased. At 120° C. the pH 2 samples were stable while the glucose levels in the neutral samples dropped slightly. Corrosion inhibitors adversely affect the assay, even when present at very low concentrations.

### Example 15

### Limits of Detection of Galactose

The general assay procedure for tests on galactose consisted of adding 50 μL of the solution to be analysed to 50 μL of working solution. 5 mL working solution was prepared from: 4.75 mL 1× reaction buffer, 100 μL galactose oxidase (100 U/mL), 100 μL horseradish peroxidise (10 U/mL), 50 μL amplex red or Amplex UltraRed (10 mM) (Invitrogen). Assays were carried out in 96-well plates and after addition of the working solution the plates were incubated at 37° C. for 30 min before analysis. The settings of the luminometer (Berthold Mithras) for analysis were as follows, lamp energy, 1000; λₑₓ 546 nm; λₑₘ 610 nm; counting time, 1 sec.

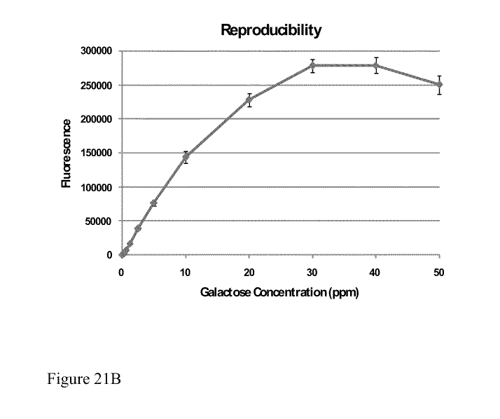

A calibration curve was generated by analysing galactose solutions prepared by serial dilution (50, 40, 30, 20, 10, 5, 2.5, 0.625, 0.3125, and 0 ppm). All concentrations quoted refer to the concentration of the solution before addition of the 50 μL of enzymes and reagents for analysis (FIG. 21A). To determine the reproducibility of the assay, these samples were rerun on three different days with freshly prepared working solution (FIG. 21B).

Results indicate that galactose can be detected within a concentration range of 0-30 ppm with a limit of detection of ca. 0.3 ppm. A linear response between 0 and 10 ppm is seen with R²=0.998. The assay was also shown to be reproducible; the graph displays the 95% confidence intervals. Further work suggested that Amplex Ultrared offered enhanced fluorescence and sensitivity and is recommended for use over Amplex Red reagent.

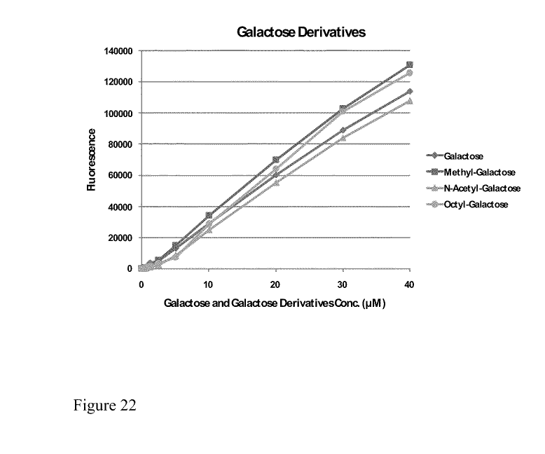

Results indicate that galactose derivatives may be used to label treatment chemicals, as they could also be detected with the assay (FIG. 22)

### Example 16

### Impact of Interferences on Galactose Assay

The effects of various potential interfering agents was investigated by preparing 2% aqueous solutions and then serial diluting to 0.2, 0.02, 0.002 and 0.0002%. Each of these solutions was added in a 50:50 ratio to 10 ppm galactose, therefore the final galactose concentration was 5 ppm. The interferences investigated using this method were scale inhibitors (2 types), a corrosion inhibitor, MEG, methanol and crude oil. Controls were prepared in which water was added in place of the galactose solution. Further controls for the scale and corrosion inhibitors and crude oil were run which did not contain any working solution (50 μL water was added instead), this was to determine the intrinsic fluorescence of these samples.

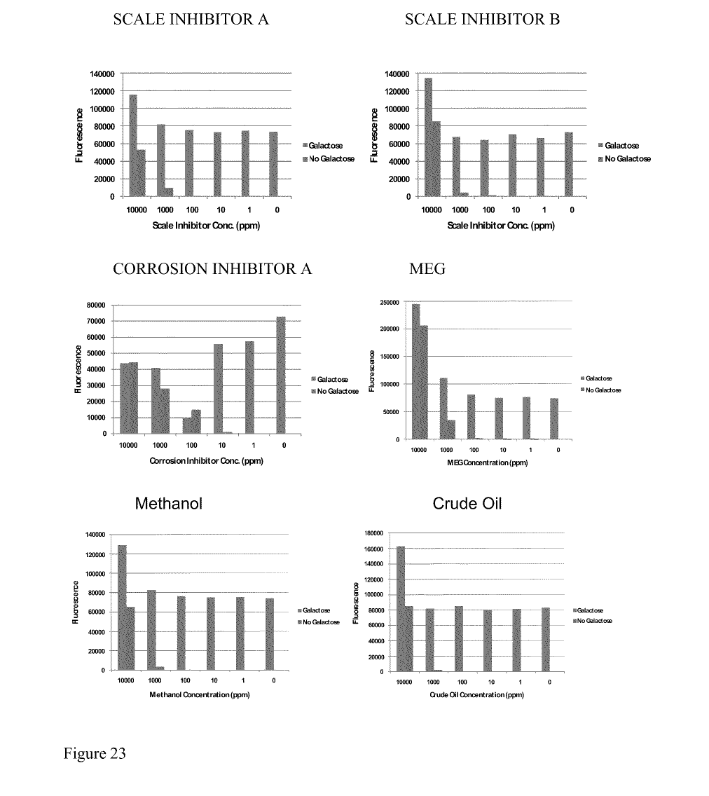

Results (FIG. 23) indicate that low concentrations of treatment chemicals (in concentrations expected in produced fluids e.g. <100 ppm scale inhibitor) do not adversely impact the assay.

### Example 17

### Effect of Other Labels on the Galactose Assay

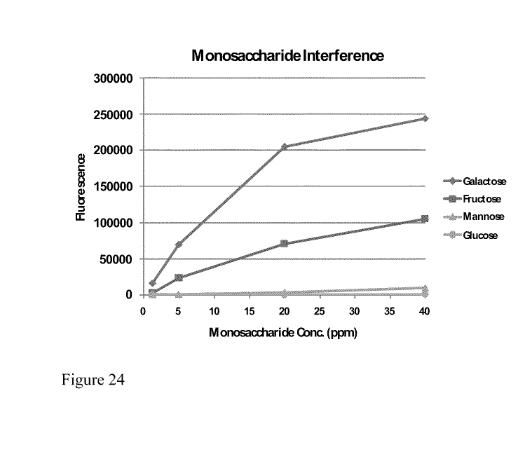

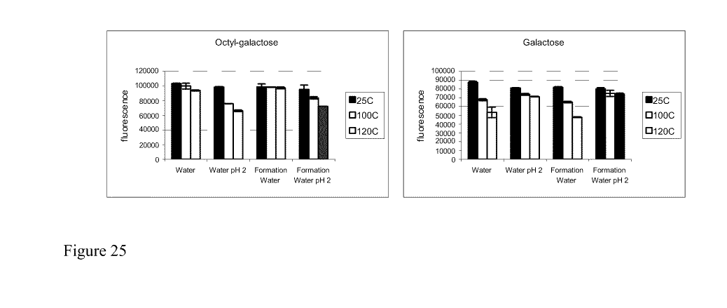

To determine if the galactose assay could function even in the presence of other labels, so enabling multiple labels to be used at once the effects of inclusion of fructose, mannose or glucose in the solution was determined Results indicate (FIG. 24) that fructose can be oxidized by galactose oxidase and would be unsuitable if used with galactose although mannose and glucose did not interfere with the assay and may be used as labels in the same system.

### Example 18

### Thermal Stability of Galactose and Derivatives

The thermal stability of both galactose and octy-galactose was investigated. Galactose and octyl-galacotse solutions (50 ppm, 30 mL) were prepared in both deionised water and formation water. These solutions were divided and the pH of one water sample and one formation water sample was adjusted to 2 with HCl. 4.5 mL of each solution was placed in a duran bottle with Teflon tape wrapped around the threads to prevent evaporation. The eight samples were heated at 100 or 120° C. for 20 h. The remaining solutions were kept at 4° C. in between experiments. Each of the samples was diluted 10-fold before analysis.

Results (FIG. 25) indicate that galactose and derivatives maybe sufficiently stable to 100° C. although a drop in concentration is observed above this temperature.

### Example 19

### Limits of Detection for Xanthin and Hypoxanthine

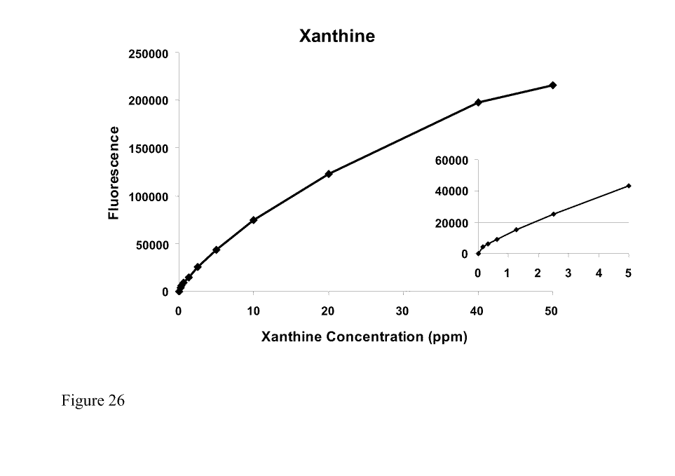

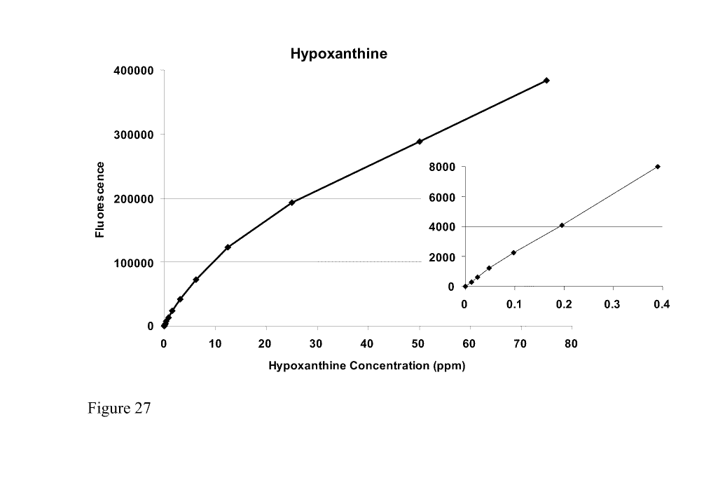

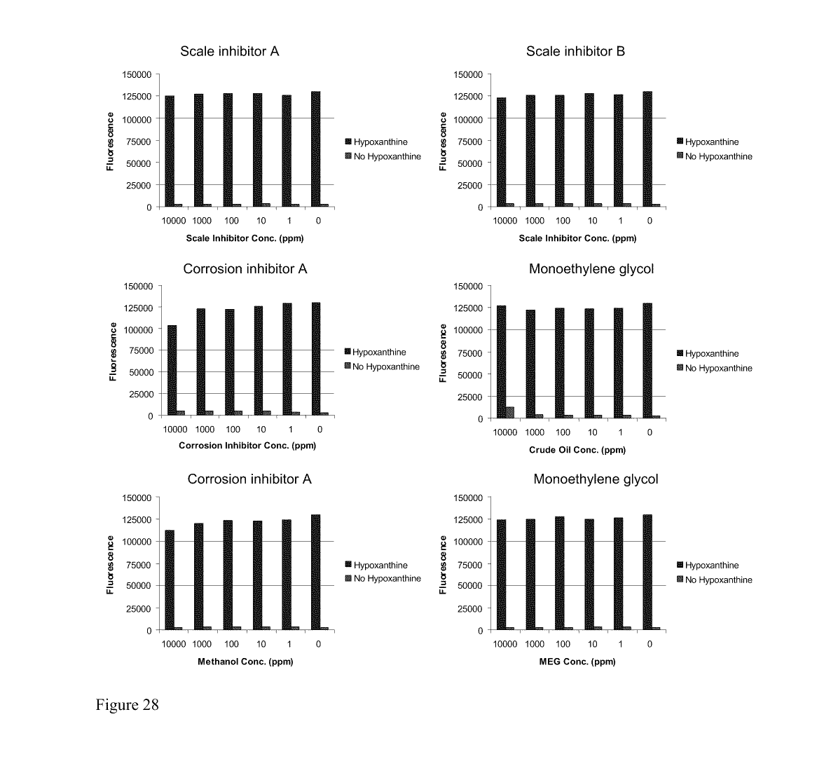

An assay for determining the concentration of xanthine and hypoxanthine is commercially available. This assay uses xanthine oxidase to catalyze the oxidation of hypoxanthine or xanthine, to uric acid and superoxide. The superoxide spontaneously degrades to hydrogen peroxide (H₂O₂), which reacts stoichiometrically with Amplex® Red in the presence of horseradishperoxidase (HRP). A fluorescent product, resorufin, is generated which can be detected fluorometrically or spectrophotometrically. Results show that the limit of detection of xanthine is less than 0.16 ppm (FIG. 26) and the limit of detection of hypoxanthine is >0.02 ppm (FIG. 27).

### Example 20

### Effect of Interferences on the Xanthine and Hypoxanthine Assay

The effects of various potential interfering agents was investigated by preparing 2% aqueous solutions and then serial diluting to 0.2, 0.02, 0.002 and 0.0002%. Each of these solutions was added in a 50:50 ratio to 12.5 ppm hypoxanthine, therefore the final hypoxanthine concentration was 6.25 ppm. The interferences investigated using this method were two scale inhibitors, a corrosion inhibitor, MEG, methanol and crude oil. Controls were prepared in which water was added in place of the hypoxanthine solution. Further controls for the scale and corrosion inhibitors and crude oil were run which did not contain any working solution (50 μL water was added instead), this was to determine the intrinsic fluorescence of these samples.

Corrosion inhibitor and methanol had an adverse affect on the assay at the highest concentrations investigated (10,000 ppm); however these levels are well above those expected in a real system (FIG. 28)

### Example 21

### Thermostability of Xanthine and Hypoxanthine

The thermal stability of both xanthine and hypoxanthine was investigated; 75 ppm solutions were prepared in deionised water. These solutions were divided and the pH of one sample of each compound was adjusted to 2 with HCl. 4.5 mL of each solution was placed in a duran bottle with Teflon tape wrapped around the threads to prevent evaporation. The samples were heated at 120° C. for 20 h. The remaining solutions were kept at 25° C. Each of the samples was diluted 10-fold to 7.5 ppm before analysis.

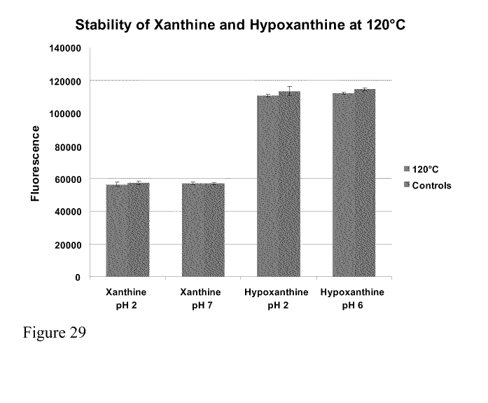

Results (FIG. 29) indicate that xanthine and hypoxanthine are stable at room temperature and 120° C. at both acidic and neutral pH.

### Example 22

### Coupling Biotin to a Corrosion Inhibitor

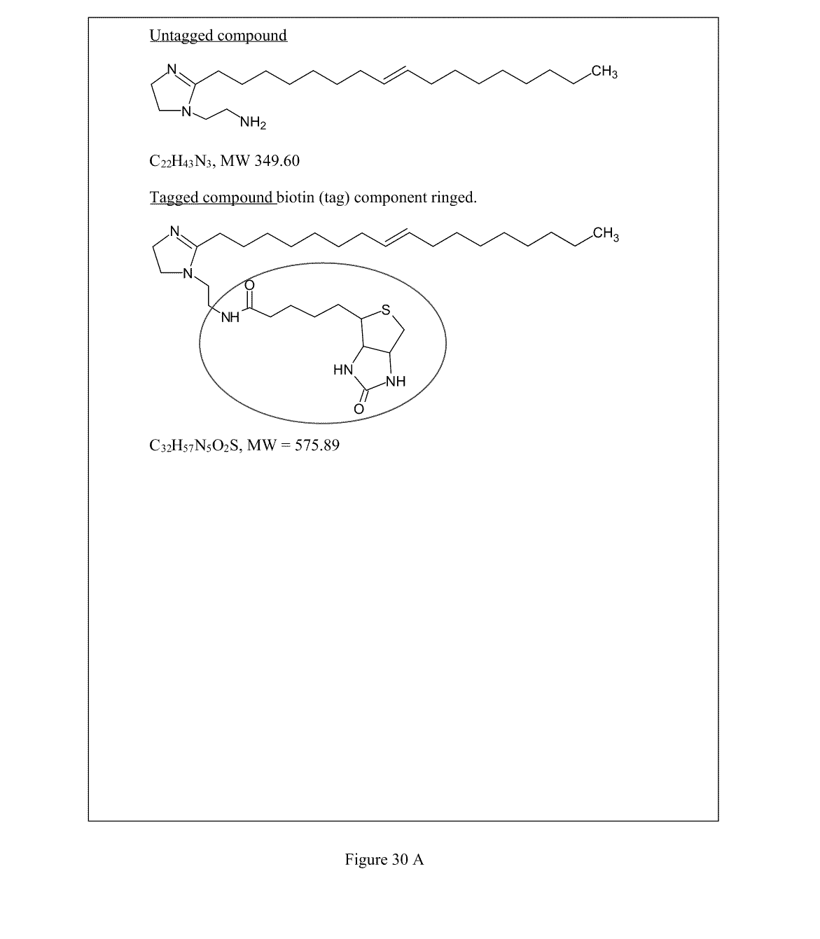

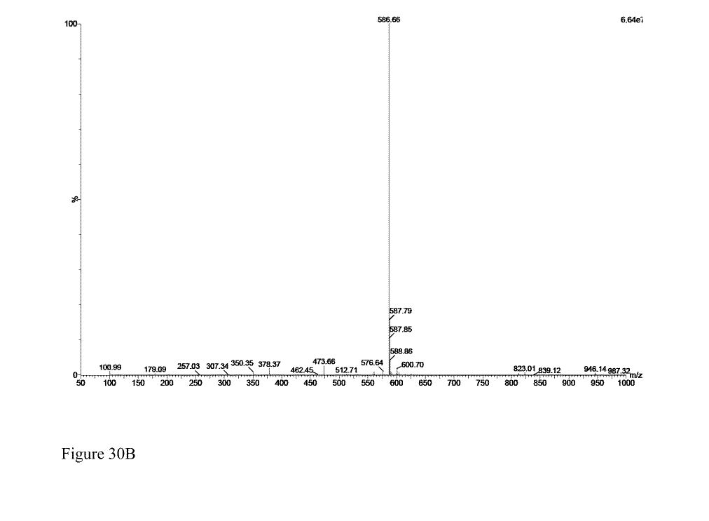

In order to produce a treatment composition comprising a label and treatment substance according to the invention, the coupling of biotin to an amino ethyl imidazole corrosion inhibitor, where the aliphatic chain is Elaidic acid, a C₁₇ fatty acid. In one example, an amide bond is formed between a primary amine and a carboxylic acid using carbodiimide based coupling chemistry, results of mass spectrometry are shown in FIG. 30B. This reaction may be performed by those skilled in the art.

The widely used HABA assay was used to determine amount of biotin present in labeled and unlabeled samples. This is a displacement assay based on the binding of biotin to avidin where HABA forms a coloured complex (with a known extinction coefficient) with avidin and when it is displaced from the avidin, it is no longer coloured. The reduction in absorbance after the addition of a biotin-containing sample to the mixture is used to determine the concentration of biotin added.

Results are shown in Table 1 and are the average of 2 replicates. Labeled samples were diluted to contain an expected 80 uM of biotin, controls were at 71 uM, this is considered an ideal range for the HABA assay. Equivalent concentrations of unlabeled corrosion inhibitor was 140 ppm and labeled corrosion inhibitor 230 ppm.

Results indicate that unlabeled samples did not interfere with the assay and that biotin was detected in the labeled samples suggesting latently detectable labels may be useful for labelling labelging corrosion inhibitors. There were some solubility issues and we believe this explains the lower than expected concentration.

TABLE 1

| | Δ | Biotin concentration |
|---|---|---|
| Sample | Abs500 | (uM) |
| Unlabeled | 0.0057 | 1.7 |
| Labeled | 0.1473 | 43.3 |

### Example 23

### Use of Labels to Capture Treatment Chemicals from Solutions and Detection

The solution to be tested may contain interferences that have a deleterious impact on detection assay e.g. presence of salts, treatment chemicals, debris, opaque solutions, background fluorescence. The chemical may also need to be separated from other similar chemicals, as may occur in comingled streams. Also, the labeled chemical may be present in multiple phases and need to be detected in both, thus corrosion inhibitors show complex partitioning behaviour and an ideal detection method is one that assesses the levels of marker in both phases. Incorporation of a capture assay would offer improvements for monitoring in these situations whereby the label may be used as a ‘hook’ that is bound by a biomacromolecule that can be used to ‘fish out’ the treatment chemical from interferences, so enabling detection and improving limits of detection.

1. A composition for treating a system for conduction and containment of fluid, the composition comprising a treatment substance associated with a label, the association between the treatment substance and the label being sufficiently stable that a detectable signal produced due to interaction of the label with a biomacromolecule is representative of the presence of the treatment substance.

2. A composition according to claim 1, wherein the label is attached to the treatment substance.

3. A composition according to claim 2, wherein the label is attached to a terminal end of the treatment substance.

4. A composition according to claim 1, wherein the label is conjugated to a polymerisation initiator.

5. A composition according to claim 1, wherein the label is conjugated to a transfer agent to create a functional transfer agent.

6. A composition according to claim 1 wherein the label is conjugated to an end-capping agent.

7. A composition according to claim 1 wherein the biomacromolecule includes a site for specific interaction with the label.

8. A composition according to claim 1 wherein the biomacromolecule and the label associate as part of molecular signalling complexes in nature.

9. A composition according to claim 1, wherein the signal generated due to the interaction between the label and the biomacromolecule is an optical signal.

10. A composition according to any claim 1, wherein the signal is generated on addition of a second molecule to a sample containing the composition and the biomacromolecule.

11-13. (canceled)

14. A composition according to claim 1, wherein the label is selected from: vitamins including biotin, selenobiotin or oxybiotin, thiamine, riboflavin, niacin (nicotinic acid), pathothenic acid, citrate, cobalamin, folic acid, ascorbic acid, retinol, vitamins C, D, E or K; luciferin; coelenterazine; chitin; amino acids such as histidine; or monosaccharides, polysaccharides and carbohydrates including arabinose, deoxyribose, lyxose, ribulose, xylose, xylulose, maltose, glucose, fructose, ribose, or trehalose, caffeine, imidazoline, steroid hormones, chlorpromazine and cAMP, cortisol, 6-ketoproslabellandins, thyroxine, triiodothyronine, anthocyanins, cholesterol, L-gulono-1,4-lactone, bile salts including cholic acid, chenodeoxycholic acid, deoxycholic and glycocholate eicosanoids (proslabellandins, prostacyclins, the thromboxanes and the leukotrienes), galactose and derivatives including 2-N-acetyle galactose, 1-methyl-beta-D-galactose, 1-octyl-beta-D-galactose, xanthine and hypoxanthine, catchetolamines such as epinephrine and norepinephrine, nucleotides such as adenine, cytosine, guanine, tyrosine, uracil, monophosphate, in diphosphate and triphosphate forms and the associated biomacromolecule is selected accordingly to the label used from; avidin and its functional analogues e.g. streptavidin, neutravidin and nitroavidin; thiamine binding-protein; riboflavin binding protein (flavoprotein); nicotinic acid binding protein; pantothenic acid binding protein; citrate binding protein, cobalamin binding protein; folic acid binding protein; ascorbic acid binding protein; retinol binding protein; vitamin D binding protein e.g. group specific protein (Gc); Vitamin E binding protein; Vitamin K binding protein; luciferase; coelenterate luciferase; chitin binding protein; histidine transporter protein; arabinose binding protein; deoxyribose binding protein; lyxose binding protein; ribulose binding protein; xylose binding protein; xylulose binding protein; maltose binding protein; glucose binding protein; fructose binding protein; ribose binding protein; trehalose binding protein or lectin; caffeine binding protein; imidazoline binding protein; steroid hormone receptors; chlorpromazine binding protein; cAMP binding protein; cortisol binding protein; 6-keto-proslabellandin antibody including labelled antibodies such as aqueorin or GFP labelled antibodies; thyroxine binding proteins including thyroxine-binding globulin, transthyretin and albumin; triiodothronine binding protein; glutathione-S-transferases; cholesterol binding proteins such as VIP21/caveolin and cholesterol oxidase; L-gulono-1,4-lactone binding proteins including Rv1771, L-gulono-1,4-lactone dehydrogenase and L-gulono-1,4-lactone oxidase; glutathione S-transferases and bile binding proteins including ileal bile acid binding proteins and liver fatty acid-binding proteins, proslabellandin receptors including PPARg, prostacyclin receptors including PTGIR and thromboxane receptors such as TXA2; L-ascorbate binding protein including L-ascorbate oxidase; receptor protein, galactose binding protein including galactose oxidase, xanthine oxidase, xanthine dehydrogenase, phosphoribosyltransferase, xanthine binding RNAs, catecholamine regulated protein (CRP40), catecholamine binding proteins, adrenergic receptors (alpha and beta), epinephrine receptor, norepinephrine receptor; nucleotide binding proteins such as G proteins and ATP binding proteins respectively.

15. A composition according to claim 1, wherein the label is detectable, in the presence of its associated biomacromolecule, by a fluorescence detector, luminescence detector, Raman detector, optical microscope, CCD camera, photographic film, fibre-optic device, photometric detector, MEMS device, single photon detector, spectrophotometer, chromatography system or by eye.

16-17. (canceled)

18. A composition according to claim 1, wherein the composition is detectable in the fluid at a concentration of at least 1 ppb when in the presence of a biomacromolecule

19. A method of manufacturing a composition for treating a system for conduction and containment of fluid, the composition comprising a treatment substance associated with a label according to claim 14, comprising:
a) mixing a treatment substance comprising polymeric scale inhibitors, phosphonate scale inhibitors, corrosion inhibitors, hydrate inhibitors, wax inhibitors, anti-fouling agents, asphaltene inhibitors, hydrogen sulphide scavengers, pH stabilizers flow additives, anti-foaming agents, detergents or demulsifiers with the label to make a reaction mixture; and
b) allowing the treatment substance and label to associate;
wherein the association that is formed between the treatment substance and the label is sufficiently stable that a detectable signal produced due to interaction of the label with a biomacromolecule is representative of the presence of the treatment substance.

20-21. (canceled)

22. A method according to claim 19, further comprising the step of removing the free label from the reaction mixture.

23-25. (canceled)

26. A method according to claim 19, in which at least one monomer unit of the treatment substance and at least one monomer unit of the label are mixed together in step (a) so that they are copolymerised in step (b) to produce a polymeric labelled composition.

27. A method of monitoring at least one composition according to claim 1 in a fluid conducting and containment system comprising:
a) adding a predetermined amount of the at least one composition to a fluid at a first location in the system;
b) adding a biomacromolecule to a fluid at a second location in the system, said second location being downstream of the first location wherein the predetermined amount of the composition at the first location is sufficient for the concentration of the composition at the second location to be above its detection limit and the concentration of the biomacromolecule is sufficient to produce a detectable change in the fluid due to a specific interaction between the label and the biomacromolecule; measuring the detectable change in the fluid;
c) measuring the detectable change in the fluid;
d) analysing any measured detectable change to determine the concentration of the label at the second location;
e) using the data obtained in step (d) in order to assess the concentration of the composition in the second location.

28. A method according to claim 27, further comprising the step of taking a sample of fluid from the second location in the system, the biomacromolecule being added to the sample.

29. A method according to claim 28 in which the sample taken is treated to improve detection of the signal, such that the sample is concentrated, bleached, filtered or immobilised to improve detection of the signal before the subsequent method steps (b-e).

30. (canceled)

31. A method according to claim 27 further comprising the step of adding a second detection molecule to the sample after or simultaneously with the addition of the biomacromolecule to the sample.

32. A method according to claim 31, wherein the second detection molecule reacts with a chemical product of the interaction between the label and the biomacromolecule, and wherein the chemical product is hydrogen peroxide.

33. (canceled)

34. A method according to claim 31, wherein the second detection molecule is Amplex Red in the presence of peroxidase; Phenol Red in the presence of peroxidase; ferrous ions in the presence of xylenol or orange; or a cyclic diacy hydrazide in the presence of peroxidase.

35. A method according to claim 27 wherein multiple treatment substances are monitored, each composition containing a different treatment substance, each treatment substance being labelled with a different label so that each different composition can be differentiated according to a different signal.

36-37. (canceled)

38. A method according to claim 27 wherein the method is performed offline, inline, atline or online.

39-43. (canceled)

44. A method of treating a fluid conducting and containment system comprising the steps of:
a) determining the concentration of a composition for treating a system for conduction and containment of fluid, the compositing comprising a treatment substance associated with a label, the association between the treatment substance and the label being sufficiently stable that a detectable signal produced due to interaction of the label with a biomacromolecule is representative of the presence of the treatment substance using the method of claim 27;
b) administering the at least one composition in order to maintain effective concentrations of said composition for treatment of the system.

45-48. (canceled)

49. A kit for use in monitoring at least one composition according to claim 1 in a system for conduction and containment of fluid, comprising;
a) at least one composition for treating a system for conduction and containment of fluid, the compositing comprising a treatment substance associated with a label, the association between the treatment substance and the label being sufficiently stable that a detectable signal produced due to interaction of the label with a biomacromolecule is representative of the presence of the treatment substance; and
b) a biomacromolecule selected accordingly to the label included in the composition.

50. A kit according to claim 49, further including means for taking a sample from said system.

51. A kit according to claim 49, further including a second detection molecule for detection of a chemical signal.

52. (canceled)
